SEQUENCE LISTING (23 sequences)

SEQ ID NO: 1 - protein, 116 aa, artificial sequence
  synthetic polypeptide (BB fragment of Staphylococcus aureus protein A)
ADNKFNKEQQNAFYEILHLPNLNEEQRNGFIQSLKDDPSQSANLLAEAKKLNDAQAPKADNKFNKEQQNAFYEILHLPNLNEEQRNGFIQSLKDDPSQSANLLAEAKKLNDAQAPK

SEQ ID NO: 2 - protein, 116 aa, artificial sequence
  synthetic polypeptide (ZZ derivative of BB fragment of Staphylococcus aureus protein A)
VDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQAPK

SEQ ID NO: 3 - protein, 9 aa, artificial sequence
  synthetic peptide (HIV Tat 49-57)
RKKRRQRRR

SEQ ID NO: 4 - protein, 11 aa, artificial sequence
  synthetic peptide (HIV Tat 28-48)
FTKKGLGISYG

SEQ ID NO: 5 - protein, 154 aa, artificial sequence
  synthetic polypeptide (R domain of Diphteria Toxin)
PGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 6 - protein, 16 aa, artificial sequence
  synthetic peptide (penetratin 43-58)
RQIKIWFQNRRMKWKK

SEQ ID NO: 7 - protein, 15 aa, artificial sequence
  synthetic peptide (Diphteria Toxin 453-467)
NGRKIRMRCRAIDGD

SEQ ID NO: 8 - protein, 21 aa, artificial sequence
  synthetic ppetide (HIV Tat 37-57)
CFTKKGLGISYGRKKRRQRRR

SEQ ID NO: 9 - protein, 36 aa, artificial sequence
  synthetic peptide (HIV Tat22-57C(22-37)S)
SNNSYSKKSSFHSQVSFTKKGLGISYGRKKRRQRRR

SEQ ID NO: 10 - protein, 101 aa, artificial sequence
  synthetic polypeptide (HIV Tat consensus sequence)
MEPVDPKLEPWKHPGSQPKTACNNCYCKKCCFHCQVCFTKKGLGISYGRKKRRQRRRAPQDSQTHQVSLSKQPASQPRGDPTGPKESKKKVERETETDPVD

SEQ ID NO: 11 - DNA, 873 bp, artificial sequence
  synthetic polypeptide (ZZ-DTRBDcoli)
atggcgcaacacgatgaagccgtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacgcgaattcgagctcggtaccgtcgacgccgggtcataaaacccagccgtttctgcatgatggctatgcggtgagctggaacaccgtggaagatagcattattcgtaccggctttcagggcgaaagcggccatgatattaaaattaccgcggaaaacaccccgctgccgattgcgggtgttctgctgccgaccattccgggcaaactggatgtgaacaaaagcaaaacccatattagcgtgaacggccgtaaaattcgtatgcgttgccgtgcgattgatggtgatgtgaccttttgccgtccgaaaagcccggtgtatgtgggcaacggcgtgcatgcgaacctgcatgtggcgtttcatcgtagcagcagcgaaaaaatccatagcaacgaaattagcagcgatagcattggcgtgctgggctatcagaaaaccgtggatcataccaaagtgaactctaaactgagcctgttcttcgaaatcaaaagcggaagctta

SEQ ID NO: 12 - protein, 291 aa, artificial sequence
  synthetic polypeptide (ZZ-DTRBDcoli)
MAQHDEAVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQAPKVDANSSSVPSTPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKSGSL

SEQ ID NO: 13 - DNA, 930 bp, artificial sequence
  synthetic polynucleotide (ZZ-DTRBDHEK )
atgggctggtcctgcatcatcctgtttctggtggccacagccaccggtgttcactctgcccagcatgatgaggccgtggacaacaagttcaacaaagagcagcagaacgccttctacgagatcctgcatctgcccaacctgaacgaggaacagagaaacgccttcatccagagcctgaaggacgaccctagccagagcgctaatctgctggccgaggccaagaagctgaacgacgctcaggcccctaaagtcgataacaagtttaacaaagaacaacaaaatgccttttatgagatcctccaccttccgaatctcaatgaggaacagcggaatgcctttattcagtccctgaaggatgatccctctcagtccgccaacctgctggctgaagctaaaaaactgaatgatgcccaggctcctaaggtggacgcgaatgctagcagcggggtaccctcaacaccagggcacaagactcagcccttcctgcacgatggctatgctgtgagctggaatacagtggaagattccatcattaggacaggctttcaaggcgaaagtggccatgacatcaagattactgccgaaaacaccccattgccgatagctggtgttctccttcccacgattcctgggaaactggacgtcaacaagtccaagactcacatctccgtgaatggacgcaaaatacggatgcgatgtagagccattgatggcgatgtgaccttttgcagacccaaatctcctgtgtacgttgggaatgtaggtcatgcgaatctccatgtcgcatttcaccgttcatcctctgagaagatccacagcaacgagatcagctctgactcaattggagtgctgggataccagaaaaccgtagaccataccaaggtcaacagcaagctgagtctgttcttcgagatcaaaagcggaagctta

SEQ ID NO: 14 - protein, 291 aa, artificial sequence
  synthetic polypeptide (ZZ-DTRBDHEK)
AQHDEAVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQAPKVDANASSGVPSTPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNVGHANLHVAFHRSSSEKIHSNEISSDSIGVLGYQKTVDHTKVNSKLSLFFEIKSGSL

SEQ ID NO: 15 - DNA, 873 bp, artificial sequence
  synthetic polynucleotide (BB-DTRBDcoli)
atggcgcaacacgatgaagccgcagacaacaaattcaacaaagaacaacaaaacgcgttctatgagatcttacatttacctaacttaaacgaagaacaacgaaacggtttcatccaaagtttaaaagatgacccaagccaaagcgctaaccttttagcagaagctaaaaagctaaatgatgctcaggcgccgaaagcagacaacaaattcaacaaagaacaacaaaacgcgttctatgagatcttacatttacctaacttaaacgaagaacaacgaaacggtttcatccaaagtttaaaagatgacccaagccaaagcgctaaccttttagcagaagctaaaaagctaaatgatgctcaggcgccgaaagtagacgcgaattcgagctcggtaccgtcgacgccgggtcataaaacccagccgtttctgcatgatggctatgcggtgagctggaacaccgtggaagatagcattattcgtaccggctttcagggcgaaagcggccatgatattaaaattaccgcggaaaacaccccgctgccgattgcgggtgttctgctgccgaccattccgggcaaactggatgtgaacaaaagcaaaacccatattagcgtgaacggccgtaaaattcgtatgcgttgccgtgcgattgatggtgatgtgaccttttgccgtccgaaaagcccggtgtatgtgggcaacggcgtgcatgcgaacctgcatgtggcgtttcatcgtagcagcagcgaaaaaatccatagcaacgaaattagcagcgatagcattggcgtgctgggctatcagaaaaccgtggatcataccaaagtgaactctaaactgagcctgttcttcgaaatcaaaagcggaagctta

SEQ ID NO: 16 - protein, 291 aa, artificial sequence
  synthetic polypeptide (BB-DTRBDcoli)
MAQHDEAADNKFNKEQQNAFYEILHLPNLNEEQRNGFIQSLKDDPSQSANLLAEAKKLNDAQAPKADNKFNKEQQNAFYEILHLPNLNEEQRNGFIQSLKDDPSQSANLLAEAKKLNDAQAPKVDANSSSVPSTPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKSGSL

SEQ ID NO: 17 - DNA, 930 bp, artificial sequence
  synthetic polynucleotide (BB-DTRBDHEK)
atgggctggtcctgcatcatcctgtttctggtggccacagccaccggtgttcactctgcccagcatgatgaggccgccgacaacaagttcaacaaagagcagcagaacgccttctacgagatcctgcatctgcccaacctgaacgaggaacagagaaacggcttcatccagagcctgaaggacgaccctagccagagcgctaatctgctggccgaggccaagaagctgaacgacgctcaggcccctaaagccgataacaagtttaacaaagaacaacaaaatgccttttatgagatcctccaccttccgaatctcaatgaggaacagcggaatggctttattcagtccctgaaggatgatccctctcagtccgccaacctgctggctgaagctaaaaaactgaatgatgcccaggctcctaaggtggacgcgaatgctagcagcggggtaccctcaacaccagggcacaagactcagcccttcctgcacgatggctatgctgtgagctggaatacagtggaagattccatcattaggacaggctttcaaggcgaaagtggccatgacatcaagattactgccgaaaacaccccattgccgatagctggtgttctccttcccacgattcctgggaaactggacgtcaacaagtccaagactcacatctccgtgaatggacgcaaaatacggatgcgatgtagagccattgatggcgatgtgaccttttgcagacccaaatctcctgtgtacgttgggaatgtaggtcatgcgaatctccatgtcgcatttcaccgttcatcctctgagaagatccacagcaacgagatcagctctgactcaattggagtgctgggataccagaaaaccgtagaccataccaaggtcaacagcaagctgagtctgttcttcgagatcaaaagcggaagctta

SEQ ID NO: 18 - protein, 291 aa, artificial sequence
  synthetic polypeptide (BB-DTRBDHEK)
AQHDEAADNKFNKEQQNAFYEILHLPNLNEEQRNGFIQSLKDDPSQSANLLAEAKKLNDAQAPKADNKFNKEQQNAFYEILHLPNLNEEQRNGFIQSLKDDPSQSANLLAEAKKLNDAQAPKVDANASSGVPSTPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNVGHANLHVAFHRSSSEKIHSNEISSDSIGVLGYQKTVDHTKVNSKLSLFFEIKSGSL

SEQ ID NO: 19 - DNA, 504 bp, artificial sequence
  synthetic polynucleotide (ZZ-Tat22-57C(22-37)Scoli)
atggcgcaacacgatgaagccgtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacgcgaattcgagctcggtacccagcaataattcctatagcaaaaaaagcagctttcatagccaggtgagctttaccaaaaaaggcctgggcattagctatggccgtaaaaaacgtcgtcagcgtcgtcgt

SEQ ID NO: 20 - protein, 168 aa, artificial sequence
  synthetic polypeptide (ZZ-Tat22-57C(22-37)Scoli)
MAQHDEAVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQAPKVDANSSSVPSNNSYSKKSSFHSQVSFTKKGLGISYGRKKRRQRRR

SEQ ID NO: 21 - protein, 11 aa, artificial sequence
  synthetic peptide (linker)
VDANSSSVPST

SEQ ID NO: 22 - protein, 12 aa, artificial sequence
  synthetic peptide (linker)
VDANASSGVPST

SEQ ID NO: 23 - protein, 9 aa, artificial sequence
  synthetic peptide (linker)
VDANSSSVP

(19) United States
(12) Patent Application Publication
(10) Pub. No.: US 2024/0199706 A1
(43) Pub. Date: Jun. 20, 2024

(54) IMMUNOMODULATORY COMPLEX AND USES THEREOF FOR THERAPY

(71) Applicant: COMMISSARIAT A L'ENERGIE ATOMIQUE ET AUX ENERGIES ALTERNATIVES, Paris (FR)

(72) Inventors: Michel LEONETTI, GIF SUR YVETTE CEDEX (FR); Alexandra SAVATIER, GIF SUR YVETTE CEDEX (FR); Celine COUSIN, GIF SUR YVETTE CEDEX (FR); Culina SLOBODAN, GIF SUR YVETTE CEDEX (FR)

(21) Appl. No.: 17/999,898

(22) PCT Filed: May 28, 2021
(86) PCT No.: PCT/EP2021/064449

§ 371 Date: Nov. 25, 2022

(30) Foreign Application Priority Data
May 28, 2020 (FR) FR20 05663

(51) Int. Cl. C07K 14/16; A61K 38/00; A61K 39/00; A61P 37/04; C07K 14/34; C07K 14/705; C07K 14/735; C07K 16/28

(52) U.S. Cl. CPC C07K 14/163; A61P 37/04; C07K 14/34; C07K 14/70535; C07K 14/7056; C07K 16/2803; A61K 38/00; A61K 2039/505

(57) ABSTRACT

The invention relates to a molecular complex consisting of at least one ligand of a sulphated sugar of the glycosaminoglycan family linked to at least one ligand of a surface molecule of antigen-presenting cells or NK or NKT cells, for use as an immunomodulatory drug, in particular in the immunotherapy of cancer and infectious diseases.

### SEQUENCE LISTING

This application includes as the Sequence Listing the complete contents of the accompanying text file “Sequence.txt”, created May 16, 2023, containing 25,676 bytes, hereby incorporated by reference.

### TECHNICAL FIELD

The invention belongs to the field of modulatory immunotherapies. The invention relates to a molecular complex consisting of at least one ligand of a sulfated sugar of the glycosaminoglycan family and at least one ligand of another surface molecule of antigen-presenting cells or of NK or NKT cells, said ligands being bonded to one another, for use as an immunomodulatory medicament, in particular in the immunotherapy of cancer and infectious diseases.

### PRIOR ART

The majority of therapeutic approaches are based on medicaments which are capable of acting directly on the target responsible for the disease or on cells exhibiting a non-physiological state. However, disease processes may be promoted by dysfunctions of the immune system. In particular, immunosuppression mechanisms are observed during tumor progression (Galon J. and D. Bruni, Nat. Rev. Drug Discov., 2019 18(3): 197-218) or during infections with an infectious pathogen (Wherry E. J. and M. Kurachi, Nature Rev. Immunol., 2015, 15(8):486-499). These observations have led to envisioning the development of immunotherapy medicaments which are capable of re-establishing adequate functioning of immune defenses, particularly by modulating the activity of T lymphocytes or NK or NKT cells, in order to enable the immune system to more effectively control disease processes. The aim of these modulatory immunotherapies is to re-establish or inhibit the functionality of a large part of a lymphocyte repertoire, particularly the repertoire of CD4+ T, CD8+ T or regulatory T lymphocytes. In this respect, they differ from vaccine immunotherapies, the aim of which is to induce a limited number of lymphocytes corresponding to specific cells of the Ag(s) included in the vaccine.

In order to develop immunotherapy medicaments, it is necessary to identify beforehand targets which have a crucial role in controlling the immune response. Over the course of the 1990s, it was demonstrated that molecules expressed at the surface of T lymphocytes, referred to respectively as PD-1 and CTLA-4, could induce inhibitory signals which downregulate the activity of these cells and could thus regulate certain immune defense mechanisms (Ishida, Y., Agata, Y., Shibahara, K., & Honjo, T. (1992). EMBO J., 11(11), 3887-3895; Freeman, et al. (2000). J Exp Med, 192(7), 1027-1034; Krummel M F, Allison J P, J. Exp. Med., 1995 Aug. 1; 182(2):459-65). These two molecules were named immune checkpoints, immune checkpoint inhibitor molecules or ICP inhibitors.

The molecular mechanisms responsible for the activity of these ICP inhibitors have been studied. In particular, it has been observed that PD-1 and CTLA-4 interact with ligands expressed at the surface of antigen-presenting cells (APC) or of tumor cells. These ligands are named, respectively, PD-L1 and B7. The PD-1/PD-L1 or CTLA-4/B7 interaction then mediates inhibitory signals from the T lymphocytes and as a result causes different immunosuppression mechanisms. In addition, it has been demonstrated that antibodies (Ab) specific of PD-1, PD-L1 or CTLA-4 could block the PD-1/PD-L1 or CTLA-4/B7 interaction, thereby making it possible to remove the inhibition and to reactivate the T lymphocytes (Hodi, F. S. et al., PNAS. (2003), 100(8), 4712-4717; Iwai, Y. et al., Int. Immunol., (2005). 17(2), 133-144). Some of these immunomodulatory Ab have proven capable of limiting the growth of various cancers (melanoma, lung cancer, etc.) and to significantly increase the life expectancy of patients. They are now commonly used as anti-tumor immunotherapy medicament in humans (Adachi K. and K. Tamada, Cancer Sci., 2015; 106(8):945-50; Riley R S et al., Nat. Rev. Drug Discov. 2019 18(3):175-196). They are also envisioned for other therapeutic fields, particularly for the treatment of infectious diseases (Rao M. et al., Int. J. Infect. Dis., 2017; 56:221-228). However, these first inhibitory anti-ICPs have the disadvantage of only working in 10 to 30% of patients suffering from cancer (Pitt J M et al., Immunity. 2016 Jun. 21; 44(6): 1255-69). Thus, there are numerous research groups aiming to discover novel immunomodulatory compounds which are more effective and/or which can be used in combination with the immunotherapies described above. Since several novel ICP inhibitors have been identified, research has focused on selecting compounds capable of binding these ICPs or their ICP-ligands and thereby neutralizing the ICP/ICP-ligand binding in order to reactivate the T lymphocytes. The discovery of ICP activators expressed at the surface of the T cell has led to other research focusing on selecting agonist ligands to these ICPs (Mahoney K M. Et al., Nat. Rev. Drug Discov., 2019, 14:561-584; De Sousa Linhares A., Front. Immunol., 2018, 31; 9:1909; Granier C. et al, ESMO Open, 2017 Jul. 3; 2 (2):e000213).

In order to immunomodulate the immune response as effectively as possible, it has been sought to select therapeutic molecules which are capable of binding ICPs or ICP-ligands expressed selectively at the surface of effector immune cells such as T lymphocytes and NK or NKT cells, or Ag-presenting cells (APC). This is because this expression selectivity makes it possible to limit the spread of the molecule toward non-immune cells. This results in an increase the therapeutic efficacy and a decrease in the risk of side effects.

The glycocalyx consists in particular of proteoglycans, which are glycoproteins comprising one or more unbranched glycosaminoglycan (GAG) chains. Among the latter, the family of heparan sulfate proteoglycans (HSPG) corresponds to proteins associated with sulfated GAGs: heparan sulfates (HS). HSPGs, which have a central role in numerous biological processes (cell proliferation, cell adhesion, inflammation, coagulation, cell penetration of pathogenic microorganisms, in particular viruses and parasites) are found at the surface of the majority of mammalian cells and in extracellular matrices (Dreyfuss et al., Annuals of the Brazilian Academy of Sciences, 2009, 81, 409-429). This ubiquitous expression therefore means that HSPGs and their HS domains are not considered to represent relevant immunomodulatory targets. Moreover, the capacity of HS ligands to induce immune cell activation has not been identified to date.

### SUMMARY OF THE INVENTION

During the course of their work, the inventors first discovered that a ligand of a sulfated sugar of the GAG family, heparan sulfates (HS), is incapable of activating dendritic cells in vitro. They then observed that a molecular complex containing this ligand and a ligand of a surface molecule of antigen-presenting cells (APC) potentiates the immunomodulatory effect of the heparan sulfate (HS) ligand. Indeed, this molecular complex induces the APC even more strongly, as shown by the increased secretion of the cytokines IL-6 and IL-12, and enables the subsequent activation of T lymphocytes. The inventors have demonstrated that the association between the ligands may be produced in the form of a covalent or non-covalent complex, in particular in the form of a fusion protein. The inventors have moreover observed that the immunomodulatory properties of this complex make it possible to better control disease processes and in particular to slow down the progression of a tumor.

These results are surprising because, although described as being able to serve as receptor or co-receptor, it had never been demonstrated that HSPGs could potentiate activation mediated by a ligand of a receptor expressed specifically at the surface of the APCs. Finally, the molecular complex which targets ubiquitously-expressed HS could spread toward the extracellular matrix or toward cells of no interest for the disease. Thus, theoretically, it should not be capable of modulating the activity of APCs and of a large repertoire of T lymphocytes with sufficient efficacy for an immunotherapeutic effect to occur.

The inventors have also discovered that the results obtained with the APCs could be transposed to other cells of the innate immune response, NK and NKT cells. Thus, the inventors have demonstrated that a ligand of a surface molecule of NK or NKT cells, incapable of inducing these cells alone, becomes capable of increasing the number of activated cells when it is associated, in a molecular complex, with a ligand of a sulfated sugar of the GAG family.

Consequently, a subject of the present invention is a molecular complex for use as immunomodulatory medicament, preferably immunostimulatory medicament, said complex consisting of at least one ligand of a sulfated sugar of the glycosaminoglycan family (first ligand or L1) and at least one ligand of another surface molecule of APC or of NK or NKT cells (second ligand or L2), said ligands being bonded to one another, and said complex being free of a specific antigen of the disease to be treated.

According to preferred embodiments of the invention, the first ligand is a heparan sulfate-binding peptide selected from the group consisting of: a peptide derived from the HIV Tat protein, comprising at least the basic region Tat 49-57 (SEQ ID NO: 3) such as the peptides Tat 49-57 (SEQ ID NO: 3), Tat37-57 (SEQ ID NO: 8) and Tat22-57C(22-37)S (SEQ ID NO:9); an R7 to R11 polyarginine peptide; and a peptide derived from the R domain of the diphtheria toxin, comprising the R domain of the diphtheria toxin (SEQ ID NO: 5) or at least the fragment DT453-467 (SEQ ID NO 7) of said domain which comprises the heparan sulfate binding region.

According to preferred embodiments of the invention, the second ligand targets a surface molecule of APCs selected from the group consisting of: C-type lectin receptors, membrane-bound immunoglobulins, receptors for the constant region of immunoglobulins, and immune checkpoint (ICP) molecules.

According to preferred embodiments of the invention, the second ligand targets a surface molecule of NK or NKT cells selected from the group consisting of: NKG2D, NKp30, NKp44, NKp46, NKp80, Ly49H receptors, KIR, NKG2A receptors, and ICP PD-1, CTLA-4, TIM-3, TIGIT, LAG-3.

The second ligand is preferably selected from the group consisting of: (i) antibodies directed against said surface molecules of the APCs or of NK or NKT cells and fragments thereof containing at least the paratope; (ii) immunoglobulins, preferably IgG, and fragments thereof comprising at least the Fc region; and (iii) proteins and protein fragments which bind to the Fc and/or Fab region of the antibodies, particularly protein A of S. aureus, the BB fragment thereof (SEQ ID NO: 1) and the ZZ derivative thereof (SEQ ID NO: 2).

According to preferred embodiments of the invention, said complex is in the form of oligomers or a mixture of monomers and oligomers.

According to preferred embodiments of the invention, said complex consists of a fusion protein between the first and the second ligand. A type of complex which is more particularly preferred according to the invention consists of a fusion protein comprising a first ligand selected from: Tat49-57 (SEQ ID NO: 3), Tat37-57 (SEQ ID NO: 8). Tat22-57C(22-37)S (SEQ ID NO 9), the R domain of the diphtheria toxin (SEQ ID NO: 5) or the fragment DT453-467 (SEQ ID NO 7) and a second ligand selected from: the BB fragment of the protein A (SEQ ID NO: 1) or the ZZ derivative thereof (SEQ ID NO: 2).

According to other preferred embodiments of the invention, the second ligand is an antibody or an antibody fragment and the first ligand forms a fusion protein with an immunoglobulin-binding element, preferably the protein A of S. aureus, the BB fragment thereof (SEQ ID NO: 1) or the ZZ derivative thereof (SEQ ID NO: 2). Preferably, said fusion protein comprises a first ligand selected from Tat49-57 (SEQ ID NO: 3), Tat37-57 (SEQ ID NO: 8), Tat22-57C(22-37)S (SEQ ID NO 9), the R domain of the diphtheria toxin (SEQ ID NO: 5) or the fragment DT453-467 (SEQ ID NO 7) and an immunoglobulin-binding element selected from the BB fragment of the protein A (SEQ ID NO: 1) or the ZZ derivative thereof (SEQ ID NO: 2). In a type of complex which is more particularly preferred according to the invention, the immunoglobulin-binding element is the BB fragment of the protein A (SEQ ID NO: 1) and said fusion protein is complexed to the second ligand which consists of a whole immunoglobulin. In another type of complex which is more particularly preferred according to the invention, said fusion protein is complexed to the second ligand which is selected from an anti-RFcgamma I, II and/or III, anti-DEC-205, anti-DC-SIGN, anti-CD74, anti-CD275, anti-CD335, anti-CD336, anti-CD56, anti-CTLA-4, anti-PD-L1, anti-OX40 antibody, or a fragment of the preceding antibodies comprising at least the paratope.

According to preferred embodiments of the invention, the complex is for use as immunostimulatory medicament, preferably for activating antigen-presenting cells, particularly dendritic cells or monocytes, for activating NK or NKT cells, and/or for activating the secretion of the cytokines IL-6 and/or IL-12.

Another subject of the present invention is a composition for use as immunomodulatory medicament, preferably immunostimulatory medicament, comprising at least one molecular complex according to the invention, and at least one pharmaceutically acceptable carrier, a carrier substance and/or an adjuvant. The adjuvant is preferably a CpG oligodeoxynucleotide, polyinosinic:polycytidylic acid or a mixture of CpG oligodeoxynucleotide(s) and of polyinosinic:polycytidylic acid, and/or the carrier substance is a nanoparticle.

According to preferred embodiments of the invention, said composition comprises at least one other therapeutic agent, preferably at least one immune checkpoint inhibitor, preferably an anti-PD-1, an anti-PD-L1 or an anti-CTLA-4.

According to preferred embodiments of the invention, the composition is for use in the immunotherapy of cancer or infectious diseases.

### DISCLOSURE OF THE INVENTION

A subject of the present invention is a molecular complex for use as immunomodulatory medicament, preferably immunostimulatory medicament, said complex consisting of at least one ligand of a sulfated sugar of the glycosaminoglycan family (first ligand) and at least one ligand of another surface molecule of APC or of NK or NKT cells (second ligand), said ligands being bonded to one another, and said complex being free of a specific antigen of the disease to be treated.

Such a complex is capable of inducing cells of the immune system favorably for triggering the immune response. Thus, when splenocytes are incubated with these complexes, the activation of dendritic cells is observed, which dendritic cells become capable of secreting IL-6 and IL-12 cytokines. Moreover, the induction of the DC can be obtained for second ligands which are incapable of triggering an effect of this kind when they are not associated, such as the isolated form of the APC ligand referred to as ZZ. The association of the HS ligand can also make it possible to potentiate the effect of a ligand which is capable, in the isolated state, of activating APCs such as a non-specific polyclonal human Ab. The association of the HS ligand with a ligand of NK or NKT cells can also make it possible to activate these cell types, whereas it is devoid of this capacity in isolated form. Finally, an immunomodulatory complex, preferably an immunostimulatory complex, according to the invention can induce a therapeutic effect, in particular an anti-tumor effect, which is superior to that obtained with an anti-ICP (anti-PD1) Ab, particularly when the composition comprises an adjuvant.

### Definitions

“Immune cells of the innate response” are intended to mean dendritic cells, NK and NKT cells, granulocytes (mastocytes, neutrophils, eosinophils and basophils) and phagocytes (monocytes, macrophages, neutrophils etc.).

“Antigen-presenting cell” (APC) is intended to mean a cell presenting Ag, a cell expressing one or more class I and class II molecules of the major histocompatibility complex (MHC) (class I and class II HLA molecules in humans) and capable of presenting the Ags to the CD4+ and CD8+ T lymphocytes specific for this Ag. As Ag-presenting cells, mention may be made in particular of dendritic cells (DC), monocytes, macrophages, B lymphocytes, lymphoblastoid cell lines, and genetically modified human or animal cell lines which express class I and class II molecules of the MHC, particularly HLA I and HLA II molecules.

“NK cell” is intended to mean a granular lymphocyte expressing the molecules CD56 and CD16 and having cytotoxic activity which does not require prior exposure to the Ag.

“NKT cell” is intended to mean a granular lymphocyte expressing NK cell markers, particularly the molecules CD56 and CD16, and T lymphocyte markers, particularly the molecule CD3. This cell has cytotoxic activity which does not require prior exposure to the Ag.

“Surface molecule of APC” is intended to mean a molecule expressed at the surface of Ag-presenting cells.

“Surface molecule of NK or NKT cell” is intended to mean a molecule expressed at the surface of NK or NKT cells.

“Surface molecule specific to APCs” is intended to mean a molecule expressed substantially on Ag-presenting cells, i.e. expressed on a highly limited number of cells other than APCs. This is therefore a molecule which has a high level of specificity of expression for APCs.

“Surface molecule specific to NK or NKT cells” is intended to mean a molecule expressed substantially on NK or NKT cells, i.e. expressed on a highly limited number of cells other than NK or NKT cells. This is therefore a molecule which has a high level of specificity of expression for NK or NKT cells.

“Glycosaminoglycan (GAG)” is intended to mean a linear polysaccharide composed of a repeating disaccharide which always contains a hexosamine (glucosamine (GlcN) or galactosamine (GaIN)) and another saccharide (glucuronic acid (GlcA), iduronic acid (IdoA), galactose (Gal)). Glucosamine is either N-sulfated (GlcNS), or N-acetylated (GlcNac). Galactosamine is always N-acetylated (GalNac). Sulfated glycosaminoglycans are in particular simple polymers of GlcA such as chondroitin sulfate and copolymers comprising residues of GlcA and/or of IdoA and/or of Gal, such as heparin, heparan sulfate, dermatan sulfate and keratan sulfate. The GAG chains can be covalently bonded to proteins (proteoglycans) which are expressed at the surface of mammalian cells and/or excreted in the extracellular matrix. The first ligand according to the invention binds to a sulfated sugar of the glycosaminoglycan family which is expressed at the surface of mammalian cells, including, inter alia, APCs and NK or NKT cells.

“Ab” is intended to mean an immunoglobulin (IgG, IgM, IgA, IgD, IgE). The term Ab denotes a specific Ab, i.e. an Ab directed against a particular molecule x (anti-molecule x Ab), particularly a surface molecule of APC (anti-APC surface molecule Ab). The term immunoglobulin denotes a non-specific Ab.

“Individual” is intended to mean a human or animal, preferably human, individual.

“Ligand” of a molecule is intended to mean any agent capable of binding this molecule with sufficiently high affinity to form a stable complex, in vitro and in vivo.

“APC ligand” is intended to mean a ligand of a surface molecule of Ag-presenting cells.

“NK or NKT cell ligand” is intended to mean a ligand of a surface molecule of NK or NKT cells. “Heparan sulfate ligand” is intended to mean an agent which binds heparin with an optical density signal at least equal to 50% of the signal measured when ZZ-Tat_{22-57C(22-37)S} is incubated at 100 nM, pH 7.2, in the ELISA assay of example 1.

“Antigen” is intended to mean any substance which can be specifically recognized by the immune system and in particular by the antibodies and cells of the immune system (B lymphocytes, CD4+ T lymphocytes, CD8+ T lymphocytes) and which is capable of inducing a specific immune response.

“Specific antigen of the disease to be treated” is intended to mean an antigen which induces an immune response directed specifically against the disease to be treated. Said specific immune response to the disease to be treated includes the production of antibodies and/or the induction of a cytotoxic T cell response (activation of CD8+ T lymphocytes) or helper T cell response (activation of CD4+ T lymphocytes) directed against an antigen of a pathogen or of a tumor cell responsible for said disease to be treated.

“Immunomodulator” is intended to mean an agent capable of controlling the immune response and in particular of upregulating or downregulating the relative response of different populations or sub-populations of immune cells such as T and B lymphocytes, APCs, NK or NKT cells. The term immunomodulator encompasses the terms immunosuppressant and immunostimulator. “Immunostimulator” is intended to mean an agent capable of upregulating, i.e. activating, the relative response of different populations or sub-populations of immune cells such as T and B lymphocytes, APCs, NK or NKT cells. The effect of the immunomodulator, preferably the immunostimulator, is exerted over a broad cellular repertoire. It is independent of the presence of a specific antigen of the disease to be treated, unlike a vaccine which requires the presence of a specific antigen of the disease to be treated and induces a specific immune response to said antigen. While the use of an immunogen or a vaccine is limited to the disease comprising the specific antigen of this disease, the immunomodulatory, preferably immunostimulatory, complex according to the invention, which is independent of the presence of a specific antigen of the disease to be treated, can be used in the immunotherapy of numerous diseases such as cancer and infectious diseases.

“Peptide” is intended to mean a sequence of natural or synthetic amino acids, optionally modified. The term peptide is used independently of the size of the amino acid sequence.

The molecular complex according to the invention consists of at least two ligands of surface molecules of APC or NK or NKT cells, bonded to one another: the first ligand, named L1, which targets a sulfated GAG, and the second ligand, named L2, which targets another surface molecule of APC. The molecular complex according to the invention may comprise one or more L1 ligands and one or more L2 ligands bonded to one another. According to certain preferred embodiments of the invention, the molecular complex consists of one L1 ligand bonded to one L2 ligand.

The molecular complex according to the invention, preferably an immunostimulatory complex, is free of a specific antigen of the disease to be treated, such as a specific vaccine antigen of the disease to be treated. If one of the ligands of the complex according to the invention comprises a specific antigen of a disease to be treated, then the treatment of said disease with said complex is excluded from the invention.

The ligands of APC, NK or NKT cells are natural, recombinant or synthetic molecules or complexes of molecules, which are protein in nature (protein, peptide, polypeptide), or lipid, carbohydrate, nucleic acid or mixed (glycolipid, glycoprotein, lipoprotein) in nature. According to certain preferred embodiments of the invention, the first and the second ligand are proteins, polypeptides or peptides, hereinafter referred to as “peptides”, which are preferably recombinant or synthetic. The recombinant proteins, polypeptides or peptides are advantageously produced in prokaryotic or eukaryotic cells in a suitable expression system, particularly suitable for the production of therapeutic proteins. For example, the recombinant proteins, polypeptides or peptides can be produced in E. coli or in HEK or CHO cells.

The first and the second ligand are associated or bonded to one another by any suitable means. They may be covalently or non-covalently bonded, either directly or via a linker, and thus form a molecular complex. The molecular complex or complex consists of two or more ligands and optionally of suitable linker(s).

The covalent association or bonding of the ligands is generated in particular by covalent chemical coupling (formation of a covalent conjugate) or by constructing a fusion protein (genetic fusion).

The immunomodulatory complex, preferably an immunostimulatory complex, is in monomeric, oligomeric or mixed form (mixture of monomers and oligomers). According to certain preferred embodiments of the invention, said complex is in oligomeric or mixed form.

According to certain preferred embodiments of the invention, the immunomodulatory molecular complex, preferably an immunostimulatory complex, consists of a fusion protein between the first ligand(s) (L1) and the second ligand(s) (L2). The amino acid sequences of L1 and L2 are fused in the suitable order, either directly or via a suitable peptide spacer. Depending on the respective sizes of the amino acid sequences of L1 and L2, they are either fused at their ends (N-terminal end of one of the sequences fused to the C-terminal end of the other sequence) or one of the sequences is inserted into the other sequence at a suitable site which does not have a deleterious effect on the binding of the ligand to the receptor thereof expressed on the surface of the APCs, NK or NKT cells.

The non-covalent bonding is generated in particular by adsorption on a nanoparticle. It may also be obtained using a molecule (linker or binding element) having a high and specific affinity for L1 or L2. This linker is covalently bonded to one of the ligands in order to non-covalently associate with the other ligand. When one of the ligands is an antibody (Ab), the linker is in particular a protein or a protein fragment which binds the Fc and/or Fab region of immunoglobulins, as described in application FR 2759296. Such immunoglobulin-binding elements include in particular the protein A of S. aureus, the BB fragment thereof (SEQ ID NO: 1) and the ZZ derivative thereof (SEQ ID NO: 2), the first two proteins binding the Fc and Fab regions of immunoglobulins, while ZZ binds only the Fc region. For example, when one of the ligands is an Ab or an antibody fragment, the immunoglobulin-binding element is covalently bonded (covalent chemical coupling or fusion protein) to the other ligand. The linker may also bind to partners which are respectively coupled to L1 and L2; for example, the linker is streptavidin which binds to biotinylated ligands L1 and L2 (L1-biotin/streptavidin/biotin-L2). The affinity of the linker for its partner in the L1-L2 complex is sufficient for it not to immediately dissociate from this complex in vivo.

According to certain preferred embodiments of the immunomodulatory complex of the invention, preferably an immunostimulatory complex, one of the ligands is an antibody or an antibody fragment and the other ligand forms a fusion protein with an immunoglobulin-binding element, preferably the protein A of S. aureus, the BB fragment thereof (SEQ ID NO: 1) or the ZZ derivative thereof (SEQ ID NO: 2). Preferably, the second ligand is an antibody or an antibody fragment and the first ligand forms a fusion protein with an immunoglobulin-binding element, preferably the protein A of S. aureus, the BB fragment thereof (SEQ ID NO: 1) or the ZZ derivative thereof (SEQ ID NO: 2).

The immunomodulatory properties, preferably immunostimulatory properties of the complex according to the invention are evaluated by conventional immunological tests known to those skilled in the art, such as those described in the examples. The immunostimulatory properties are in particular evaluated by analyzing the expansion and/or activation of different populations or sub-populations of immune cells such as CD4+ T lymphocytes, CD8+ T lymphocytes and B lymphocytes, monocytes, dendritic cells including conventional dendritic cells (CDC) and plasmacytoid dendritic cells (pDC), NK cells, NKT cells. The expansion is analyzed in particular using flow cytometry by means of suitable markers for the different cell populations to be analyzed. The activation of the immune cells may be analyzed by detecting markers of activation (CD69) and/or of maturation (CD86) using flow cytometry or by assaying cytokines secreted in the extracellular medium, in particular IL-6 and IL-12, by conventional assays such as ELISA. As mentioned above, the immunomodulatory effect, preferably immunostimulatory effect, of the molecular complex according to the invention is independent of the presence of a specific antigen of the disease to be treated.

The molecular complex according to the invention, preferably an immunostimulatory complex, binds to at least one sulfated sugar of the glycosaminoglycan family expressed at the surface of APCs, NK or NKT (sulfated GAG) and to another surface molecule of APC, NK or NKT, in particular a surface molecule specific to APCs, NK or NKT. The sulfated GAG which is targeted by the first ligand is preferably a heparan sulfate, a chondroitin sulfate, a dermatan sulfate or a keratan sulfate, preferably a heparan sulfate. The sulfated GAG ligand (first ligand) may be derived from a mammalian cell or a pathogenic microorganism, particularly a virus (adenovirus, cytomegalovirus, HIV, Sindbis virus), a bacterium (Mycobacterium bovis, Bordetella pertussis), a parasite (Leishmania sp.) or a toxin; in particular, it is a molecular complex of a molecule or a fragment thereof which binds heparin and or heparan sulfates. Such ligands are described in particular in “Heparan Binding Proteins”, H. Edward Conrad, Academic Press, San Diego and London and Dreyfuss et al., Annuals of the Brazilian Academy of Sciences, 2009, 81, 409-429 (see in particular table II). Examples of these ligands include, nonlimitingly: endogenous GAG ligands (thrombin, urokinase, vitronectin, fibroblast growth factor and others), the HIV Tat protein (SEQ ID NO: 10) and fragments thereof, in particular fragments comprising solely the Tat basic region (Tat49-57 (SEQ ID NO: 3) or the Tat basic region and central region (core; Tat38-48 (SEQ ID NO: 4)); dodecahedra derived from the adenovirus penton (Vivès et al., Virology, 2004, 321: 332-340); the HIV envelope protein or the V3 region of this protein (Roderiquez et al., J. Virol., 1995, 69, 2233-), the envelope glycoprotein of the Sindbis virus (Byrnes, A. P. et Griffin, D. E., J. Virol., 1998, 2, 7349-7356) and the R domain of the diphtheria toxin (DTR or DTRBD; fragment 382 to 535 of DT (SEQ ID NO: 5)); Lobeck et al., Infection and Immunity, 1998, 66, 418-423). Mention may also be made of cell penetrating peptides (CPP) which bind heparin, heparan sulfates and/or chondroitin sulfates, such as peptides which are highly rich in basic residues, in particular in arginines, which include the abovementioned peptides derived from the basic region of the HIV Tat protein (Tat49-57), and polyarginine (R7 to R11) peptides, and also basic/amphiphilic peptides such as peptides derived from the homeodomain of the antennapedia protein (penetratin; fragment 43-58 (SEQ ID NO: 6)).

Alternatively, the first ligand is a natural or recombinant Ab directed against a sulfated GAG, preferably heparin, heparan sulfates or chondroitin sulfates, or a fragment of this Ab containing at least the paratope (Ag-binding domain) such as a Fab. Fab′, F(ab′)₂, Fv or single-chain Fv (scFv), Fabc fragment, and Fab fragment comprising a portion of the Fc domain. Such Ab are described in particular in Thompson et al., J. Biol. Chem., 2009, 284, 35621-35631 and van Kuppevelt et al., J. Biol. Chem., 1998, 273, 12960-12966. Said Ab or Ab fragment is preferably human or humanized.

According to advantageous embodiments of the molecular complex, preferably immunostimulatory molecular complex, of the invention, the first ligand is a heparan sulfate-binding peptide selected from the group consisting of: a peptide derived from the HIV Tat protein, comprising at least the basic region Tat 49-57 (SEQ ID NO: 3) such as the peptides Tat 49-57 (SEQ ID NO: 3), Tat37-57 (SEQ ID NO: 8) and Tat22-57C(22-37)S (SEQ ID NO 9); an R7 to R11 polyarginine peptide; and a peptide derived from the R domain of the diphtheria toxin, comprising the R domain of the diphtheria toxin (SEQ ID NO: 5) or at least the fragment DT453-467 (SEQ ID NO 7) of said domain which comprises the heparan sulfate binding region. The first ligand is preferably a heparan sulfate-binding peptide selected from the group consisting of: a peptide derived from the HIV Tat protein, comprising at least the basic region Tat 49-57 (SEQ ID NO: 3), in particular the peptide Tat22-57C(22-37)S (SEQ ID NO 9), and a peptide comprising the R domain of the diphtheria toxin (SEQ ID NO: 5).

In advantageous embodiments of the molecular complex, preferably immunostimulatory molecular complex, of the invention, when the complex comprises the R domain of the diphtheria toxin (DTR: SEQ ID NO: 5), then the domain is produced in mammalian cells, in particular in the form of a fusion protein with the second ligand or an immunoglobulin-binding element, so as to form oligomers.

The other molecule expressed at the surface of the APCs which is targeted by the second ligand is just as well a ubiquitous surface molecule other than a sulfated GAG; a surface molecule expressed substantially on immune cells of the innate or adaptive response including APCs, i.e. a surface molecule specific to immune cells of the innate or adaptive response including APCs; or a surface molecule expressed substantially on APCs, and in particular on dendritic cells, i.e. a surface molecule specific to APCs and in particular dendritic cells. Among these surface molecules expressed substantially on APCs and in particular on dendritic cells, mention may be made in particular of: class II MHC molecules, in particular the α and β chains and the γ chain or invariant chain (Ii, Ii fragment or CD74) of MHC II molecules; surface immunoglobulins or membrane-bound immunoglobulins; integrins such as, in particular, CD11c and MAC1, transferrin receptors, type-C lectin receptors such as, in particular, the mannose receptor (CD206), DEC-205 (CD205), CD206, DC-SIGN (CD209), LOX1, Dectin-1 (beta-glucan receptor), Dectin-2, Clec9A, Clec12A, DCIR2, FIRE and CIRE; receptors for the constant region of immunoglobulins (FcR or RFc), in particular FcγR such as FcγRI (CD64), FcγRII (CD32) and FcγRIII (CD16); the superfamily of TNF receptors such as CD40; and complement receptors. Among the surface molecules expressed substantially on APCs, mention may be made of immune checkpoint (ICP) molecules and ligands thereof (ICP-ligands) expressed on APCs, such as, nonlimitingly, PDL1, PDL2, CD155, CD80, CD86, CD40, OX40L, ICOSL (CD275), CD70 (Wykes M N, Nat. Rev. Immunol., 2018, 18:91-104).

The other molecule expressed at the surface of the NK or NKT cells which is targeted by the second ligand is just as well a ubiquitous surface molecule other than a sulfated GAG; a surface molecule expressed substantially on immune cells of the innate or adaptive response including NK or NKT cells, i.e. a surface molecule specific to immune cells of the innate or adaptive response including NK or NKT cells; or a surface molecule expressed substantially on NK or NKT cells, i.e. a surface molecule specific to NK or NKT cells. Among these surface molecule expressed substantially on NK or NKT cells, mention may be made in particular of: NKG2D, NKp30, NKp44, NKp46, NKp80, CD56, CD16, KIR, NKG2A receptors, and ICP PD-1, CTLA-4, TIM-3, TIGIT, LAG-3 and OX40.

According to advantageous embodiments of the molecular complex, preferably immunostimulatory molecular complex, of the invention, the second ligand targets a molecule expressed substantially on APCs and in particular on dendritic cells, i.e. a surface molecule specific to APC and in particular to dendritic cells, preferably selected from the group consisting of: C-type lectin receptors, membrane-bound immunoglobulins, receptors for the constant region of immunoglobulins.

According to other advantageous embodiments of the molecular complex, preferably immunostimulatory molecular complex, of the invention, the second ligand targets a surface molecule of APCs selected from the group consisting of: C-type lectin receptors, membrane-bound immunoglobulins, receptors for the constant region of immunoglobulins (RFc or FcR), and immune checkpoint (ICP) molecules and ligands thereof (ICP ligands).

According to advantageous embodiments of the invention, the second ligand targets a surface molecule expressed substantially on NK or NKT cells, i.e. a surface molecule specific to NK or NKT cells, preferably selected from the group consisting of: NKG2D, NKp30, NKp44, NKp46, NKp80, CD56, CD16, KIR, NKG2A receptors, and ICP PD-1, CTLA-4, TIM-3, TIGIT, LAG-3 and OX40; preferably NKp44 (CD336), NKp46 (CD335), NCAM (CD56), CTLA-4 and OX40.

The second ligand is in particular selected from saccharides which bind C-type lectin receptors; immunoglobulins and fragments thereof comprising the constant region which bind FcRs; proteins or protein fragments which bind the Fc region and/or Fab region of membrane-bound immunoglobulins, as described in application FR 2759296, particularly the protein A of S. aureus, the BB fragment thereof (SEQ ID NO: 1) and the ZZ derivative thereof (SEQ ID NO: 2), preferably r ZZ (SEQ ID NO: 2). Alternatively, the second ligand is an antibody directed against these surface molecules of APC, NK or NKT cells, or a fragment of this antibody containing at least the paratope (Ag-binding domain) such as Fab, Fab′, F(ab′)2, Fv or single-chain Fv (scFv), Fabc fragments, and Fab fragment comprising a portion of the Fc domain.

The antibody or antibody fragment is in particular directed against a surface molecule specific to immune cells of the innate or adaptive response, including APCs, NK and NKT cells, such as, nonlimtingly, PDL1, PDL2, CD155, CD80, CD86, CD40, OX40L, ICOSL (CD275), CD70; PDL1, PDL2, CD155, CD80, CD86, CD40, OX40L, ICOSL (CD275), CD70, PD-1, CTLA-4, TIM-3, TIGIT, LAG-3; preferably PDL1 and ICOSL (CD275) or PDL1, ICOSL (CD275), CTLA-4 and OX40.

The antibody or antibody fragment can also be directed against a surface molecule specific to APCs and in particular to dendritic cells such as an anti-RFcgamma (I, II and/or III) or anti-C-type lectin receptor antibody, in particular anti-DEC-205 or anti-DC-SIGN (CD209); anti-DEC-205, anti-DC-SIGN (CD209) or anti-invariant chain Ii (CD74). The antibody may be an agonist or an antagonist of said surface molecule; for example, the antibody is an agonist of an activator surface molecule or an antagonist of an inhibitor surface molecule. Said antibody or antibody fragment is preferably human or humanized. Such antibodies are well known to those skilled in the art, and are commercially available.

According to advantageous embodiments of the molecular complex, preferably immunostimulatory molecular complex, the second ligand is selected from the group consisting of: (i) antibodies directed against said surface molecules of antigen-presenting cells or of NK or NKT cells and fragments thereof containing at least the paratope, such as Fab. Fab′, F(ab′)2, Fv, scFv, Fabc and Fab fragments with at least one portion of the Fc domain; (ii) immunoglobulins, preferably IgG, and fragments thereof comprising at least the Fc region; and (iii) proteins and protein fragments which bind to the Fc and/or Fab region of the antibodies, particularly protein A of S. aureus, the BB fragment thereof (SEQ ID NO: 1) and the ZZ derivative thereof (SEQ ID NO: 2).

The antibodies directed against said surface molecules of antigen-presenting cells are preferably selected from anti-RFcgamma (I, II and/or III), anti-DC-SIGN (CD209), anti-DEC-205, anti-CD206, anti-CD40 antibodies; anti-RFcgamma (I, II and/or III), anti-DC-SIGN (CD209), anti-DEC-205, anti-CD206, anti-CD40, anti-invariant chain Ii (CD74), and anti-ICOSL (CD275) antibodies; preferably, anti-DC-SIGN (CD209) and anti-DEC-205 antibodies or anti-DC-SIGN (CD209), anti-DEC-205, anti-invariant chain Ii (CD74), and anti-ICOSL (CD275) antibodies.

The antibodies directed against said surface molecules of NK or NKT cells are preferably selected from anti-CD16, anti-CD56, anti-CD335, anti-CD336, anti-NKp30, anti-NKG2D, anti-NKp80, anti-Ly49H, anti-NKG2A, anti-PD-1, anti-CTLA-4, anti-TIM-3, anti-TIGIT, anti-LAG-3 and anti-OX40 antibodies; preferably, anti-NCAM (CD56), anti-Nkp46 (CD335), anti-Nkp44 (CD336), anti-CTLA-4 and anti-OX40 antibodies.

The additional ligands of the molecular complex, preferably an immunostimulatory molecular complex, are advantageously selected from sulfated GAG ligands and ligands of APC surface molecules as defined above.

A first type of preferred immunostimulatory complex according to the invention consists of a sulfated GAG ligand peptide as defined above (first ligand) associated with a protein or a protein fragment which binds the Fc and/or Fab region of immunoglobulins such as the BB fragment of the protein A of Staphylococcus aureus and the ZZ derivative thereof (second ligand), preferably in the form of a fusion protein comprising the first and the second ligand. A first type of particularly preferred complex consists of a fusion protein comprising a first ligand selected from Tat49-57 (SEQ ID NO: 3), Tat37-57 (SEQ ID NO: 8), Tat22-57C(22-37)S (SEQ ID NO 9), the R domain of the diphtheria toxin (DTR: SEQ ID NO: 5) or the fragment DT453-467 (SEQ ID NO 7); and a second ligand selected from the BB fragment of the protein A (SEQ ID NO: 1) or the ZZ derivative thereof (SEQ ID NO: 2), preferably the ZZ derivative thereof (SEQ ID NO: 2). The first and the second ligand are fused, either directly or via a suitable spacer peptide. In this first type of particularly preferred complex, the second ligand is preferably at the N-terminal end, and the first ligand at the C-terminal end, of the fusion protein; the first and second ligand are separated by a suitable spacer peptide, particularly selected from SEQ ID NO: 21 to 23. More preferably, said complex comprises one of the sequences SEQ ID NO: 12, 14, 16, 18 and 20. When the complex comprises the R domain of the diphtheria toxin (DTR: SEQ ID NO: 5), then the domain is advantageously produced in mammalian cells, in particular in the form of a fusion protein with the second ligand, so as to form oligomers.

A second type of preferred immunostimulatory complex according to the invention consists of: (i) a sulfated GAG ligand peptide as defined above (first ligand) covalently bonded to an immunoglobulin-binding element as defined above, in particular a protein or a protein fragment which binds the Fc and/or Fab region of Ab, preferably solely the Fab region, such as the protein A of Staphylococcus aureus and the BB fragment thereof, preferably in the form of a fusion protein comprising the first ligand and the immunoglobulin-binding element, said first ligand, associated covalently with the immunoglobulin-binding element, being complexed to (ii) an immunoglobulin, preferably an IgG, or a fragment comprising at least the Fc region (second ligand). Preferably a whole immunoglobulin, preferably a whole IgG. The first ligand and the immunoglobulin-binding element are fused, either directly or via a suitable spacer peptide. A second type of particularly preferred complex consists of a fusion protein comprising a first ligand selected from Tat49-57 (SEQ ID NO: 3), Tat37-57 (SEQ ID NO: 8), Tat22-57C(22-37)S (SEQ ID NO 9), the R domain of the diphtheria toxin (DTR: SEQ ID NO: 5) or the fragment DT453-467 (SEQ ID NO 7) and an immunoglobulin-binding element comprising the BB fragment of the protein A (SEQ ID NO: 1), said fusion protein being complexed to a whole immunoglobulin. In this second type of particularly preferred complex, the immunoglobulin-binding element (BB) is preferably at the N-terminal end, and the first ligand at the C-terminal end, of the fusion protein; the immunoglobulin-binding element (BB) and the first ligand are separated by a suitable spacer peptide, particularly selected from SEQ ID NO: 21 to 23. More preferably, said complex comprises one of the sequences SEQ ID NO: 16 or 18. When the complex comprises the R domain of the diphtheria toxin (DTR: SEQ ID NO: 5), then the domain is advantageously produced in mammalian cells, in particular in the form of a fusion protein with the immunoglobulin-binding element, so as to form oligomers.

A third type of preferred immunostimulatory complex according to the invention consists of: a sulfated GAG ligand peptide as defined above (first ligand), associated with an Ab selected from the group consisting of an anti-RFcgamma (I, II, and/or III) Ab, an anti-DEC-205 Ab, an anti-DC-SIGN (CD209) Ab, an anti-invariant chain Ii (CD74) Ab, an anti-ICOSL (CD275) Ab, an anti-NKp46 (CD335) Ab, an anti-NKp44 (CD336) Ab, an anti-NCAM (CD56) Ab, an anti-CTLA-4 Ab, an anti-PDL1 Ab, an anti-OX40 Ab, and a fragment of the above Ab comprising at least the paratope. Preferably, the sulfated GAG ligand peptide as defined above (first ligand) is covalently bonded to an immunoglobulin-binding element as defined above, in particular a protein or a protein fragment which binds the Fc and/or Fab region of immunoglobulins such as the BB fragment of the protein A of Staphylococcus aureus and the ZZ derivative thereof, preferably in the form of a fusion protein of the first ligand with the immunoglobulin-binding element. The first ligand and the immunoglobulin-binding element are fused, either directly or via a suitable spacer peptide. A third type of particularly preferred complex consists of a fusion protein comprising a first ligand selected from Tat49-57 (SEQ ID NO: 3), Tat37-57 (SEQ ID NO: 8), Tat22-57C(22-37)S (SEQ ID NO 9), the R domain of the diphtheria toxin (DTR: SEQ ID NO: 5) or the fragment DT453-467 (SEQ ID NO 7) and an immunoglobulin-binding element comprising the BB fragment of the protein A (SEQ ID NO: 1) or the ZZ derivative thereof (SEQ ID NO: 2), said fusion protein being complexed to an anti-RFcgamma (I, II and/or III), anti-DEC-205, anti-DC-SIGN, anti-CD74, anti-CD275, anti-CD335, anti-CD336, anti-CD56, anti-CTLA-4, anti-PDL1, anti-OX40 antibody, or to a fragment of the preceding antibodies comprising at least the paratope. In this third type of particularly preferred complex, the immunoglobulin-binding element (BB or ZZ) is preferably at the N-terminal end, and the first ligand at the C-terminal end, of the fusion protein; the immunoglobulin-binding element (BB or ZZ) and the first ligand are separated by a suitable spacer peptide, particularly selected from SEQ ID NO: 21 to 23. More preferably, said complex comprises one of the sequences SEQ ID NO: 12, 14, 16, 18 or 20. When the complex comprises the R domain of the diphtheria toxin (DTR: SEQ ID NO: 5), then the domain is advantageously produced in mammalian cells, in particular in the form of a fusion protein with the immunoglobulin-binding element, so as to form oligomers. Preferably, said complex comprises an anti-DEC-205, anti-DC-SIGN, anti-CD74, anti-CD275, anti-CD335, anti-CD336, anti-CD56, anti-CTLA-4, anti-PDL1 or anti-OX40 antibody.

According to preferred embodiments of the invention, said complex is used as an immunostimulant, preferably for activating antigen-presenting cells, particularly dendritic cells or monocytes, for activating NK or NKT cells, and/or for activating the secretion of the cytokines IL-6 and/or IL-12.

Another subject of the invention is an immunomodulatory composition, preferably an immunostimulatory composition, comprising at least one immunomodulatory complex, preferably immunostimulatory complex, according to the invention, and at least one pharmaceutically acceptable carrier, a carrier substance and/or an adjuvant.

The pharmaceutically acceptable carriers are those conventionally used.

The adjuvants are the adjuvants for humoral and/or cellular immunity which are conventionally used in immunotherapy. The adjuvants are advantageously selected from the group consisting of: oily emulsions, mineral substances, bacterial extracts, saponin, alumina hydroxide, monophosphoryl lipid A, squalene and TLR ligands, in particular oligodeoxynucleotides comprising at least one CpG sequence (CpG oligodeoxynucleotide), which are TLR9 ligands, or polyinosinic:polycytidylic acid (poly(I):poly(C) or poly I:C), which is a TLR3 ligand. According to preferred embodiments of the invention, said composition comprises at least one adjuvant, preferably a CpG oligodeoxynucleotide, polyinosinic:polycytidylic acid or a mixture of CpG oligodeoxynucleotide(s) and polyinosinic:polycytidylic acid.

The carrier substances are those which are conventionally used. They are in particular unilamellar or multilamellar liposomes, ISCOMs, virosomes (virus-like particles), saponin micelles, solid microspheres of saccharide (poly(lactide-co-glycolide)) or auriferous nature, and nanoparticles. According to preferred embodiments of the invention, said composition comprises at least one carrier substance, for example a nanoparticle or a mixture of nanoparticles.

The immunomodulatory composition, preferably immunostimulatory composition, according to the invention, comprises a complex comprising 2 ligands or more or a mixture of different complexes, said complex(es) optionally being bonded to one another by covalent or non-covalent bonds and/or incorporated within, or at the surface of, a particle such as a liposome, a virosome or a nanoparticle.

According to particular embodiments of the invention, the composition comprises a polynucleotide or a mixture of polynucleotides encoding ligands of APC, NK or NKT which are proteins, polypeptides or peptides. The polynucleotide consists of a recombinant, synthetic or semi-synthetic nucleic acid which can be expressed in cells of the host to whom the composition is administered. The nucleic acid may be a DNA, an RNA, particularly an mRNA, a mixed nucleic acid (DNA/RNA) and may be modified. For example, said composition comprises a polynucleotide comprising a sequence encoding a fusion protein comprising at least the coding sequences of the first and the second ligand, suitably fused in-frame, and optionally a sequence encoding a linker as defined above. Alternatively, the composition comprises a mixture of polynucleotides comprising at least a first polynucleotide comprising a sequence encoding the first ligand and a second polynucleotide comprising a sequence encoding the second ligand, said first or second nucleotide also comprising a sequence encoding a linker as defined above. Said polynucleotide(s) are preferably inserted in one or more expression vectors comprising suitable transcription-regulating and/or translation-regulating sequences (promoter, transcription activator, transcription terminator, polyadenylation signal) for the expression of the first ligand and of the second ligand, and optionally other ligands in vivo in the individuals to whom the composition is administered. Numerous vectors which can be used in therapy are known per se. Use may be made, inter alia, of viral vectors (adenovirus, retrovirus, lentivirus, AAV) and non-viral vectors (naked DNA), particularly a plasmid, into which the sequence of interest has previously been inserted. Alternatively, said polynucleotide(s) are mRNA, preferably modified. The use of mRNA in therapy is well-known to those skilled in the art (reviewed for example by Drew Weissman, Expert Reviews of Vaccines, October 2014, 1-17, doi: 10.1586/14760584.2015.973859).

According to particular embodiments of the invention, the composition comprises cells modified by the composition according to the invention. For example, the cells are modified by a polynucleotide, a mixture of polynucleotides or a vector as defined above, or loaded with a ligand complex as defined above. The cell is in particular a natural antigen-presenting cell such as a dendritic cell, or an artificial antigen-presenting cell such as exosomes derived from dendritic cells, or vesicles derived from cells expressing the ligands of the molecular complex according to the invention. For example, the cells are antigen-presenting cells of an individual to be treated, particularly dendritic cells which are modified ex vivo before being re-administered to the individual (ex vivo cell therapy).

The immunomodulatory composition may further comprise at least one other therapeutic agent, particularly an anti-cancer agent, anti-infective agent, another immunomodulatory agent or a vaccine antigen specific to the disease to be treated, said vaccine antigen being advantageously associated with a carrier substance or included in a suitable vector. According to preferred embodiments of the invention, said composition further comprises at least one immune checkpoint inhibitor (Marin-Acevedo J. et al., 2018, Hematol. Oncol., 11:39) such as, nonlimitingly, an anti-PD-1, an anti-PDL-1 or an anti-CTLA4, particularly an antibody, preferably a monoclonal antibody, directed against the molecule PD-1, PDL-1 or CTLA4, preferably the human molecule hPD-1, hPDL-1 or hCTLA4. The composition according to the invention advantageously comprises an anti-PD-1, in particular a monoclonal anti-PD-1 antibody, preferably anti-hPD-1. According to other preferred embodiments of the invention, said composition further comprises at least one vaccine antigen specific to the disease to be treated, preferably associated with a carrier substance or included in a suitable vector.

The immunomodulatory composition, preferably immunostimulatory composition, comprises an effective dose of complex(es), polynucleotide(s), vector(s), cell(s) which is sufficient to induce an immune response capable of producing a therapeutic effect on the disease to be treated, i.e. of reducing the symptoms of this disease. This relates in particular to attenuating the consequences of the action of a pathogen (infectious or non-infectious) or else reducing the growth of a tumor, in an individual treated with this composition. This dose is determined and adjusted based on factors such as the age, sex and weight of the subject. The immunomodulatory composition, preferably immunostimulatory composition, according to the invention, is in a pharmaceutical form suited to the chosen administration. The composition is generally administered following the customary immunotherapy protocols, at doses and for a duration which are sufficient to induce an effective immune response against the disease to be treated. The administration may be intratumoral, subcutaneous, intramuscular, intravenous, intradermal, intraperitoneal, oral, sublingual, rectal, vaginal, intranasal, by inhalation or by transdermal application. The composition is in a pharmaceutical form suited to a chosen administration.

The polynucleotides isolated or inserted into a plasmid vector are introduced into the individual to be treated, either using physical methods such as electroporation, or by associating them with any substance(s) making it possible to pass the plasma membrane, such as transporters, for example nanotransporters, liposomes, lipids or cationic polymers. Furthermore, it is advantageously possible to combine these methods, for example using electroporation associated with liposomes.

The immunomodulatory composition, preferably immunostimulatory composition, according to the present invention, is used in immunotherapy, in particular anti-tumor or anti-infective immunotherapy. The immunomodulatory composition, preferably immunostimulatory composition, according to the present invention, is used preventively or curatively, i.e. for preventing a disease in individuals or for treating individuals suffering from a disease. According to preferred embodiments of the invention, said composition is used curatively, i.e for treating individuals suffering from a disease. It may be used in combination with other treatments, whether therapeutic or surgical, particularly in combination with other therapeutic agents as defined above, the composition according to the invention and the other therapeutic agents being able to be administered simultaneously, separately or sequentially.

According to preferred embodiments of the invention, said composition is for use in the treatment of cancer. The cancers are any type of cancer which might benefit from immunotherapy, such as, nonlimitingly: breast, colon, prostate, esophageal, stomach, lung, ENT (ear, nose and throat), skin, ovarian, uterine, brain, liver or kidney cancers.

According to other preferred embodiments of the invention, said composition is used in the treatment of infectious diseases, particularly those for which the infectious agents remain in the body. The infectious diseases are any type of infectious disease which might benefit from immunotherapy (Wykes M N et al., Nat. Rev. Immunol., 2018, 18:91-104) such as, nonlimitingly: viral, bacterial, fungal or parasitic infections, particularly infections by HIV, HBV, HCV, Mycobaterium tuberculosis or Plasmodium falciparum. When said complex comprises Tat or a Tat fragment as defined above, then said composition is used in the treatment of infectious diseases other than HIV infection (AIDS). When said complex comprises DTR or a DTR fragment as defined above, then said composition is used in the treatment of infectious diseases other than diphtheria (infection by the diphtheria bacillus, Corynebacterium diphtheriae).

The immunomodulatory composition, preferably immunostimulatory composition, according to the present invention, is used either in conventional therapy or in cell therapy, or else by a combination of the two approaches.

Cell therapy comprises the preparation of antigen-presenting cells, particularly dendritic cells, or NK or NKT cells, by a conventional protocol comprising isolating peripheral blood mononuclear cells (PBMCs) from a patient to be treated and culturing the dendritic, NK or NKT cells in the presence of molecular complex(es), polynucleotide(s), vector(s) as defined above. In a second step, the antigen-presenting cells, NK or NKT loaded with said molecular complex(es) or modified by said polynucleotide(s) or vector(s) are reinjected into the patient.

Another subject of the present invention is an immunotherapy method, in particular anti-tumor or anti-infective method, characterized in that it comprises administering an immunomodulatory composition, preferably immunostimulatory composition as defined above, to an individual by any suitable means as defined above.

Another subject of the present invention is the use of an immunomodulatory composition, preferably immunostimulatory composition as defined above for preparing a medicament intended for immunotherapy, preferably anti-tumor or anti-infective immunotherapy.

The complexes of ligands of APC, NK or NKT cells according to the invention are prepared by conventional techniques known to those skilled in the art, namely:
the ligands of surface molecules of APC, NK or NKT cells can be produced by chemical synthesis or by expression of a recombinant DNA in a suitable eukaryotic or prokaryotic cell system. The peptides or proteins can be synthesized in solid phase, according to the Fmoc technique originally described by Merrifield et al. (J. Am. Chem. Soc., 1964, 85, 2149-) and purified by reverse-phase high-performance liquid chromatography. The polypeptides and proteins can be produced from the corresponding cDNA, cloned in a suitable eukaryotic or prokaryotic expression vector; the polypeptides or proteins produced in the cells modified by the recombinant vector are purified by any suitable means, particularly by affinity chromatography. Ab directed against surface molecules of APCs, NK or NKT cells are well-known and commercially available. For example, nonlimitingly, the anti-CD205 (#555831); anti-CD206 (#555952); anti-CD209 (#551186); anti-HLA-DR (#555556) Ab are available from BECTON-DICKINSON, while the anti-CD56 Ab is available from Biolegend (#304622) and the anti-CD335 (AM31284AF-N) and anti-CD336 (#AM50346PU-N) Ab are available from Origene. Alternatively, monoclonal Ab can be produced by conventional techniques known to those skilled in the art. For example, monoclonal Ab are produced from hybridomas obtained by fusing B lymphocytes, from an animal immunized with the APCs surface molecule, with myelomas, according to the technique of Köhler et Milstein (Nature, 1975, 256, 495-497); the hybridomas are cultured in vitro, particularly in fermenters, or produced in vivo in the form of ascites; alternatively, said monoclonal Ab are produced by genetic engineering as described in the U.S. Pat. No. 4,816,567. The humanized Ab are produced by general methods such as those described in International Application WO 98/45332. The Ab fragments are produced from cloned V_{H} and V_{L} regions, from mRNA of hybridomas or splenic lymphocytes from an immunized animal; for example the Fv, scFv or Fab fragments are expressed at the surface of filamentous phages according to the technique of Winter et Milstein (Nature, 1991, 349, 293-299); after several selection steps, the Ab fragments specific to the Ag are isolated and expressed in a suitable expression system by conventional techniques for cloning and expressing recombinant DNA. The Ab or the fragments thereof as defined above are purified by conventional techniques known to those skilled in the art such as affinity chromatography.

The covalent association of the first ligand (L1) to the second ligand (L2) of a surface molecule of APCs or NK or NKT cells can be produced by constructing a fusion protein in which the nucleotide sequences encoding L1 and L2 are fused in-frame in the suitable order, either directly or via a nucleotide sequence encoding a suitable spacer peptide. Depending on the respective sizes of the amino acid sequences of L1 and L2, they are either fused at their ends (N-terminal end of one of the sequences fused to the C-terminal end of the other sequence) or one of the sequences is inserted into the other sequence at a suitable site which does not have a deleterious effect on the binding of the ligand(s) to the receptor thereof expressed on the surface of the APCs or NK or NKT cells. Alternatively, the ligand(s) may be coupled covalently by any suitable means. The coupling is produced via reaction groups which are initially present or introduced into the ligand(s) beforehand. The coupling may in particular be produced at amino acid residues, the side chain of which comprises a reactive function. Among these amino acids, mention may be made of polar amino acids comprising a function as follows: —OH [serine (S), threonine (T) or tyrosine (Y)], —SH [cysteine (C)], —NH2 [lysine (K) or arginine (R)], —COOH [aspartic acid (D) or glutamic acid (E)], and polar amino acids having a side chain functionalized by adding a reactive function, particularly a chloroacetyl or bromoacetyl which reacts which thiol groups or a hydrazine group which reacts with aldehydes. The ligand is coupled by any suitable means; these means, which are known to those skilled in the art, include in particular coupling using homobifunctional reagents such as glutaraldehyde or dithiobis(succinimidyl propionate). Preferably, the coupling is produced using heterobifunctional reagents, particularly m-maleimidobenzoyl-N-hydroxysuccinimide (SMCC) or sulfo-SMCC, which each contain a maleimide group capable of reacting with free thiols. In this case, the SMCC is covalently bonded beforehand to an amine function present on the ligand. At the same time, another heterobifunctional reagent (such as N-succinimidyl-S-acetylthioactate which contains a thio-ester group which can be cleaved with hydroxylamine, or succinimidyl-pyridyl-dithiopropionate which contains a disulfide bridge which can be reduced under mild conditions), is associated with an amine function of the second partner which is one of the ligands. The second partner is subsequently treated with hydroxylamine or with a reducer in order to enable the release of the thiol. The thiolated compound is then incubated with the compound which has maleimide incorporated therein, and the coupling is obtained by reaction of the thiol group on the maleimide group. This type of covalent coupling is described in particular in Léonetti et al., J. Exp. Med., 1999, 189, 1217-1228. It is also possible to release a thiol group already present on one of the compounds, and to then couple it to another compound which has been modified beforehand using SMCC. This method, which is often employed to couple Ab to ligands, is described in particular in Ishikawa et al., J. Immunoassay, 1983, 4, 209-327.
the non-covalent complexes are prepared by bringing the second ligand (L2) into contact with the first ligand (L1) under conditions which enable the two partners to interact. This interaction may involve a linker, particularly a protein or peptide, which has high and specific affinity for one of the partners of the complex (L1 or L2). In particular, the affinity of the linker for this partner in the complex is sufficient for it not to immediately dissociate from this complex in vivo. When one of the ligands is an immunoglobulin, the linker is an immunoglobulin-binding element as described in Application FR 2759296. For example, an immunoglobulin-binding element is covalently bonded to L1 so as to form a non-covalent complex with L2.
the polynucleotides according to the invention are obtained by conventional methods which are known per se, according to standard protocols known to those skilled in the art. For example, they may be obtained by amplifying a nucleic acid sequence by PCR or RT-PCR, by screening genomic DNA libraries by hybridization with a homologous probe, or else by total or partial chemical synthesis. The recombinant vectors are constructed and introduced into host cells by conventional recombinant DNA and genetic engineering methods which are known per se.

Unless otherwise indicated, the implementation of the invention uses conventional methods of immunology, of cell culture, of cellular biology, of molecular biology and of recombinant DNA which are known to those skilled in the art.

### BRIEF DESCRIPTION OF THE DRAWINGS

Other features, details and advantages of the invention will become apparent on reading the following detailed description which refers to exemplary implementations of the present invention, and on analyzing the appended drawings, in which:

FIG. 1

FIG. 1 shows the molecular weight and degree of homogeneity of molecular complexes produced in E. coli or in HEK cells. The proteins ZZ-DTRBD_{HEK}, ZZ-DTRBD_{coli}, BB-DTRBD_{HEK}, BB-DTRBD_{coli} and ZZ-Tat_{22-57C(22-37)S} were analyzed by electrophoresis under denaturing conditions. The proteins were deposited in the presence of molecular weight markers. After electrophoretic migration, the proteins were stained with Coomassie blue.

FIG. 2

FIG. 2 shows the binding of various molecular complexes to heparin. Series dilutions of the proteins ZZfree, ZZ-DTRBD_{HEK}, ZZ-DTRBD_{coli}, BB-DTRBD_{HEK}, BB-DTRBD_{coli} and ZZ-Tat_{22-57C(22-37)S} were respectively incubated at pH 7.2 in microtitration plate wells, previously adsorbed with rabbit IgG. After 4 hours, the plates were washed and biotinylated heparin was added. After 30 minutes of incubation, the binding of the heparin to the plates was detecting using streptavidin coupled to peroxidase and a substrate of this enzyme (ABTS). The heparin binding is considered to be significant when the optical density signal is at least equal to 50% of the signal measured when ZZ-Tat_{22-57C(22-37)S} is incubated at 100 nM, pH 7.2, on the microtitration plates.

FIG. 3

FIG. 3 shows the cells types bonded by the fusion protein ZZ-DTRBD_{HEK} in a population of mouse splenocytes. Splenocytes were incubated in the presence or absence of a fixed amount of ZZ-DTRBD_{HEK} (100 nM). A series of fluorescent Ab specific to the CD11 b+pDC, cDC and CD8+ cDC (A); CD4+ T lymphocytes, CD8+ T lymphocytes, B lymphocytes, monocytes (B) was then added. After 30 minutes at 4° C., the cells were washed and analyzed by flow cytometry. The percentage of cells bonded by ZZ-DTRBD_{HEK} in the sub-population in question is shown.

FIG. 4

FIG. 4 shows that ZZ-DTRBD_{HEK} induces the secretion of IL-6 and of IL-12 by splenocytes. A fixed amount of ZZ, DTRBD or ZZ-DTRBD_{HEK} (1 μM) was incubated with mouse splenocytes. After 24 h, the supernatants were taken off and the presence of IL-6 and IL-12 was evaluated by enzyme immunoassay.

FIG. 5

FIG. 5 shows that ZZ-Tat_{22-57C(22-37)S} induces the secretion of IL-6 and of IL-12 by splenocytes. A fixed amount of ZZ, Tat_{CY49-57}, ZZ+Tat_{CY49-57} or ZZ-Tat_{22-57C(22-37)S} (1 μM) was incubated with mouse splenocytes. After 24 h, the supernatants were taken off and the presence of IL-6 and IL-12 was evaluated by enzyme immunoassay.

FIG. 6

FIG. 6 shows that the proteins ZZ-DTRBD_{HEK}, BB-DTRBD_{HEK} induce the secretion of larger amounts of IL-6 and IL-12 by splenocytes than ZZ-DTRBD_{coli} et BB-DTRBD_{coli}. A fixed amount of ZZ-DTRBD_{HEK}, ZZ-DTRBD_{coli}, BB-DTRBD_{HEK} and BB-DTRBD_{coli} (1 μM) was incubated with mouse splenocytes. After 24 h, the superatants were taken off and the presence of IL-6 and IL-12 was evaluated by enzyme immunoassay.

FIG. 7

FIG. 7 shows that ZZ-DTRBD_{HEK} induces the secretion of IL-6 and of IL-12 by isolated murine dendritic cells. Dendritic cells were isolated by magnetic sorting. The dendritic cells were then incubated in the presence or absence of a fixed amount of ZZ-DTRBD_{HEK} (0.6 UM). After 24 h, the supernatants were taken off and the presence of IL-6 and IL-12 was evaluated by enzyme immunoassay.

FIG. 8

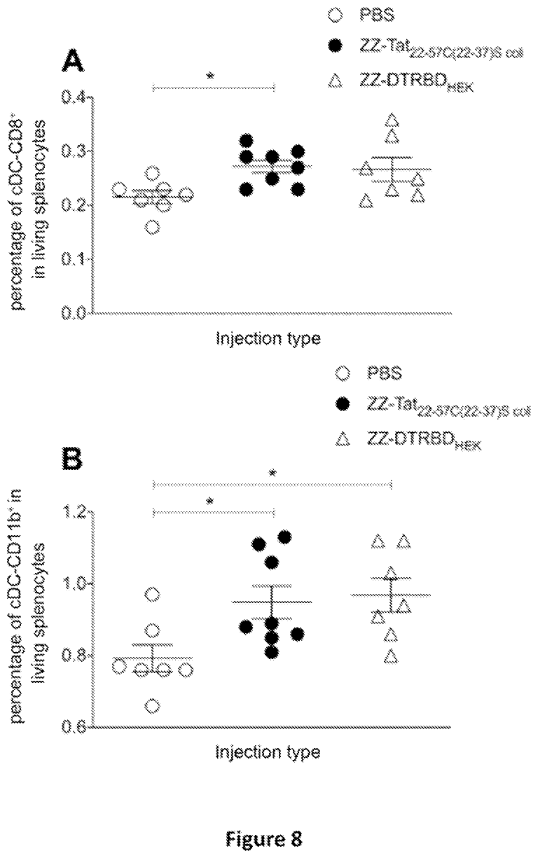

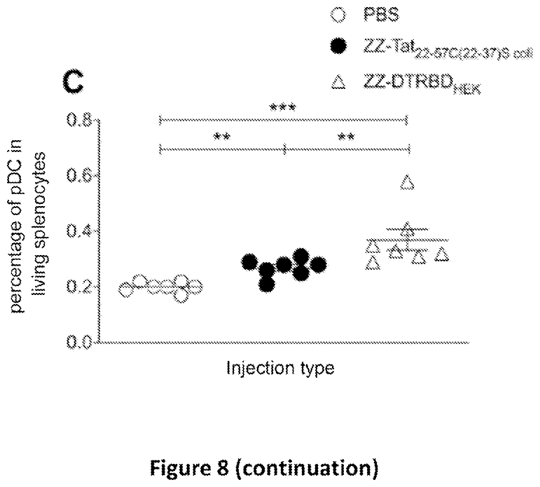

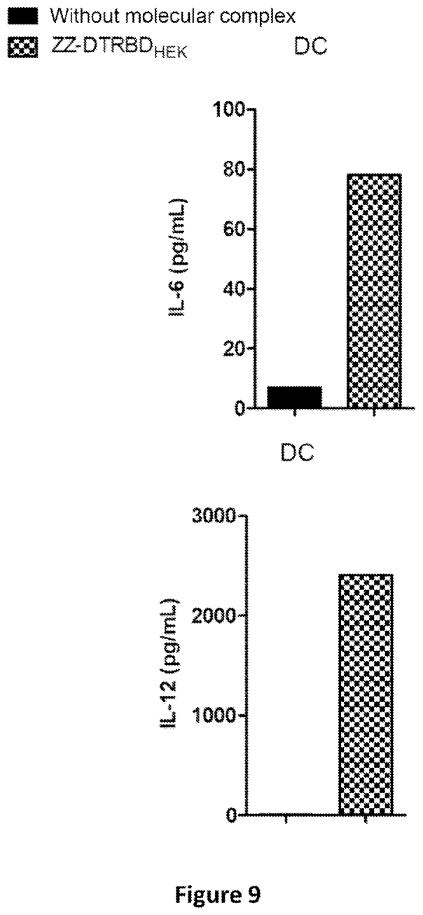

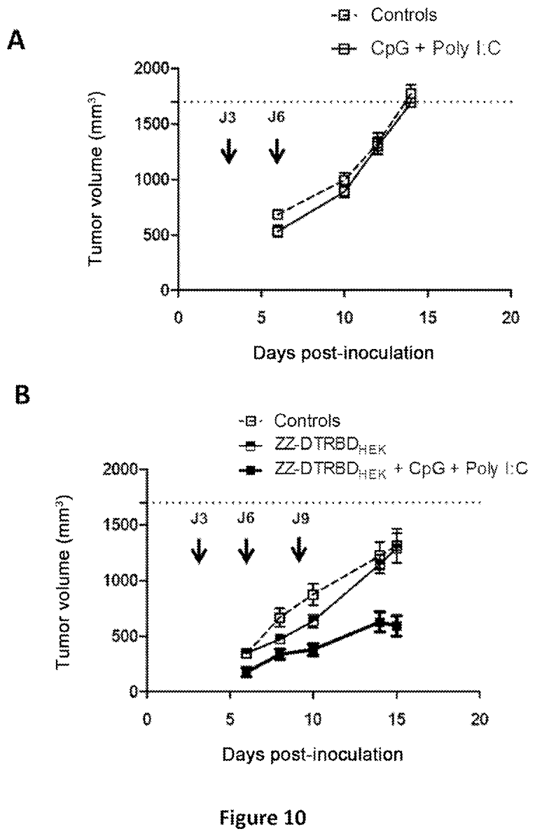

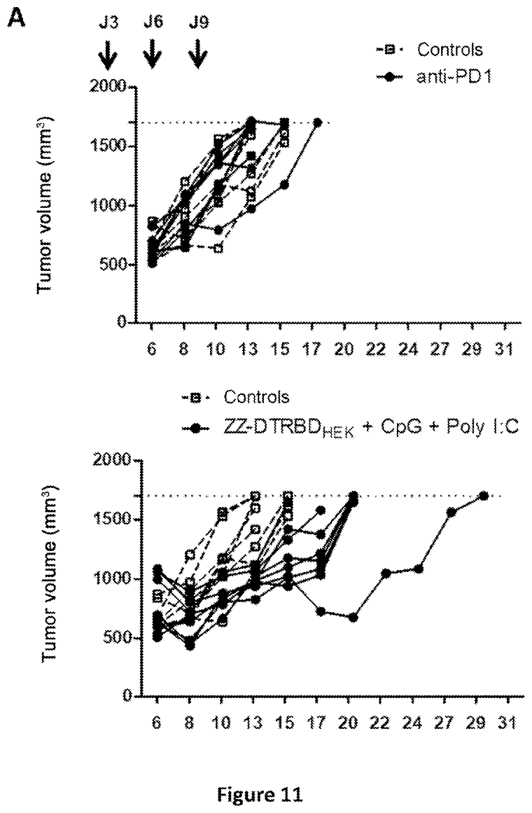

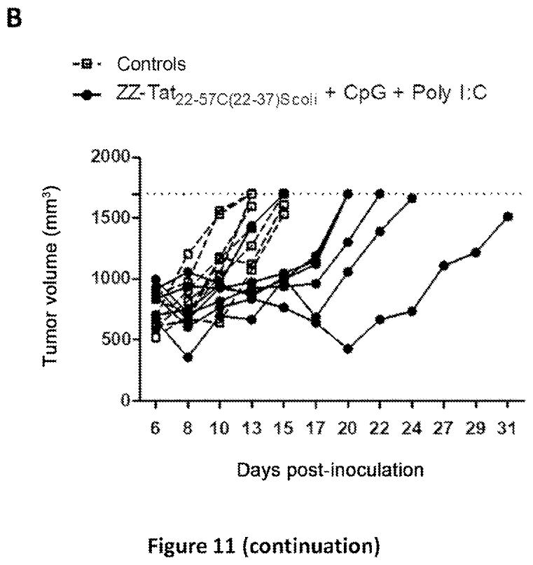

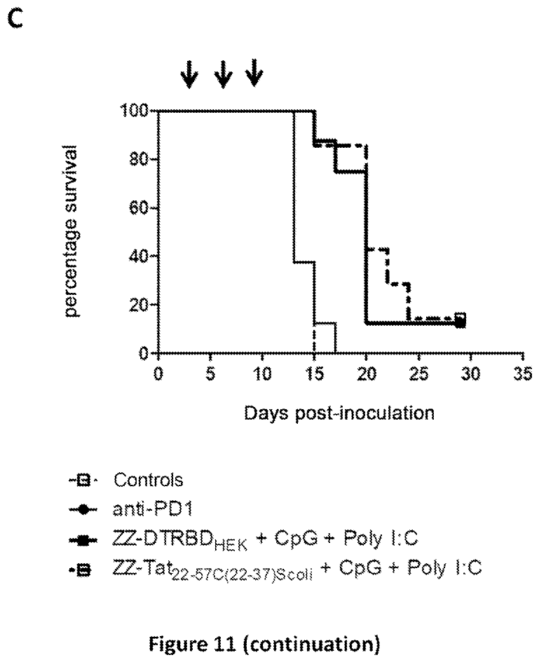

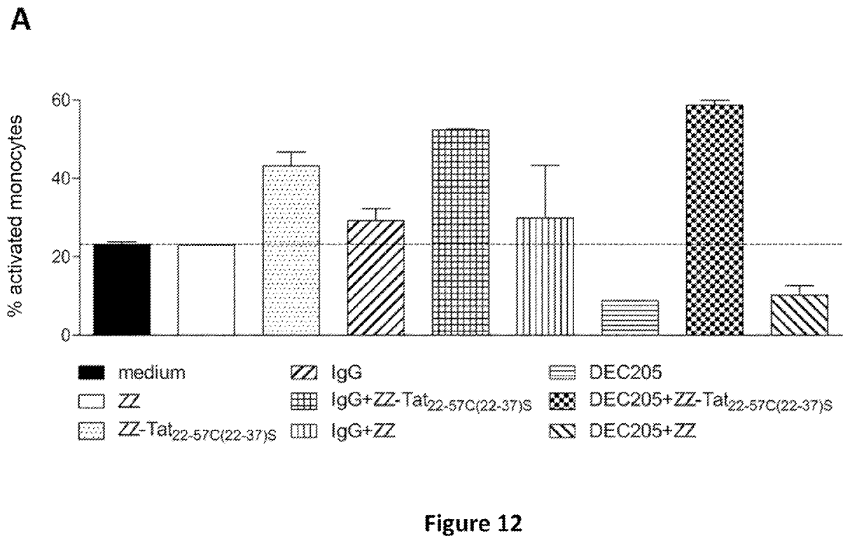

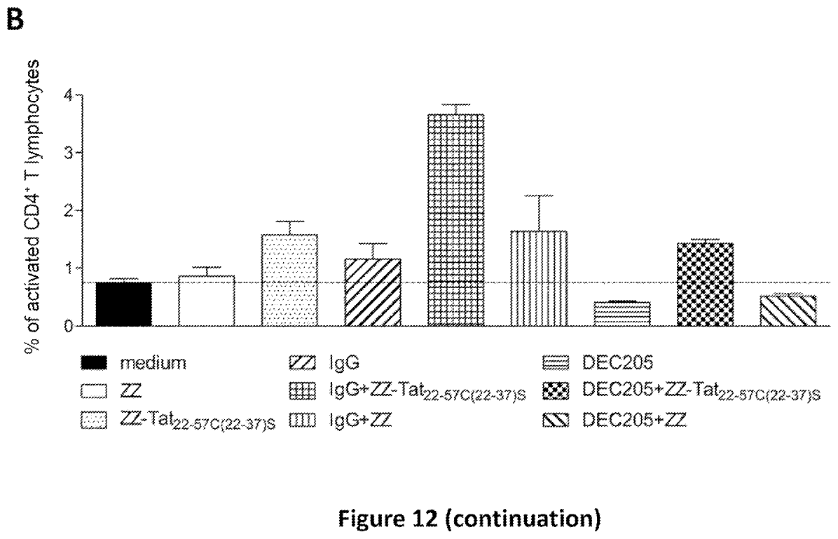

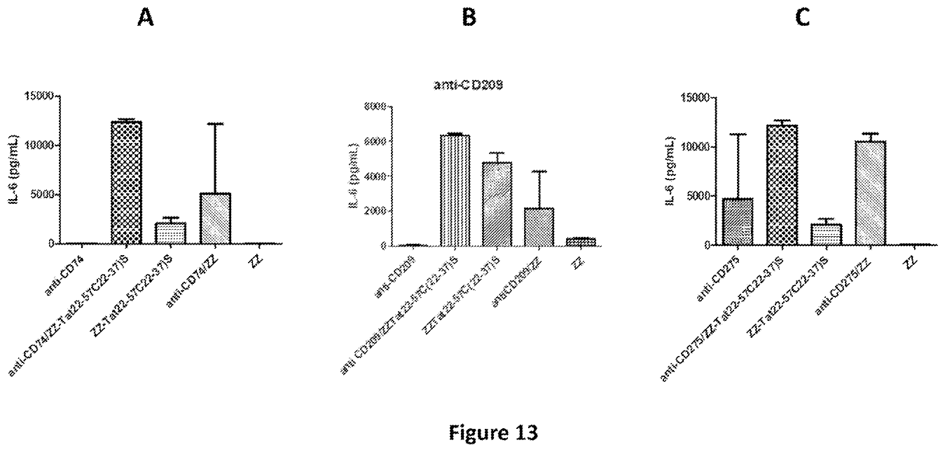

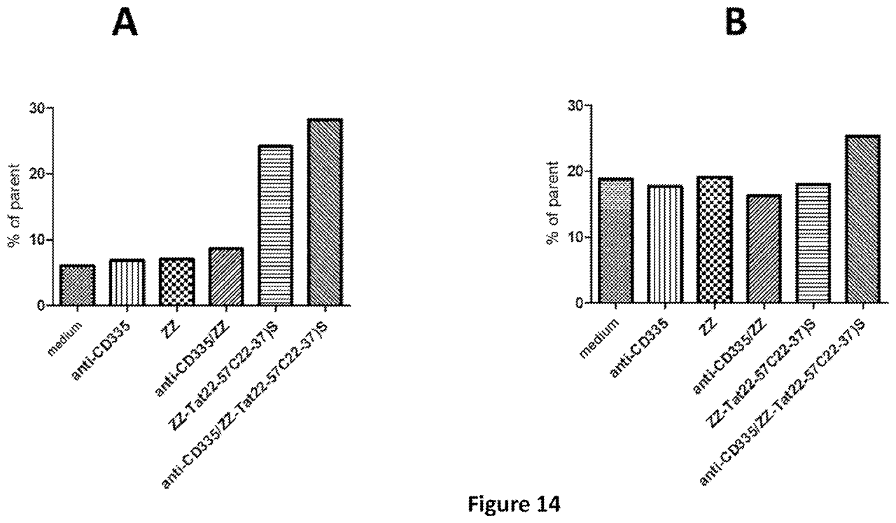

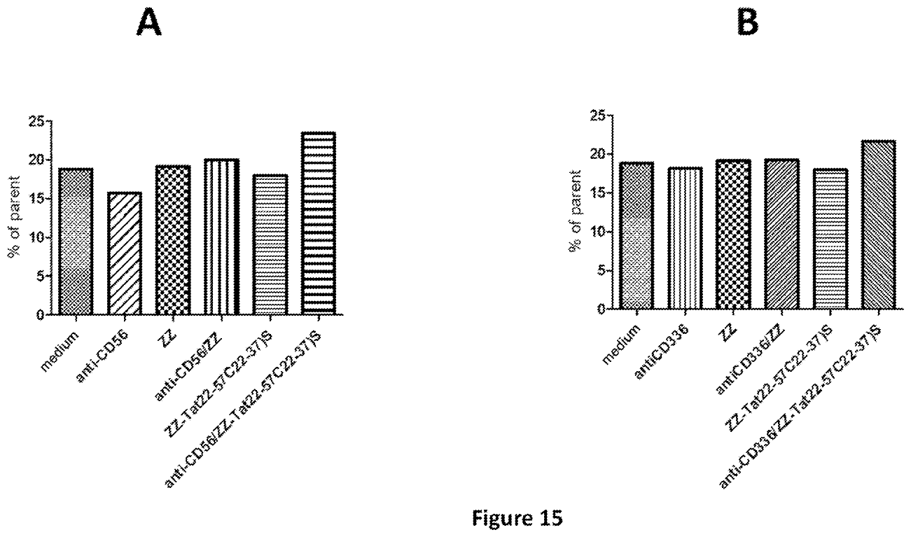

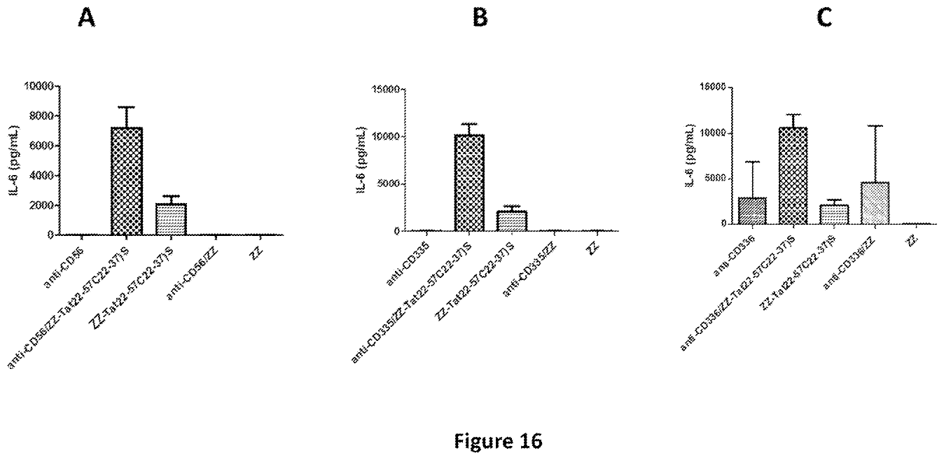

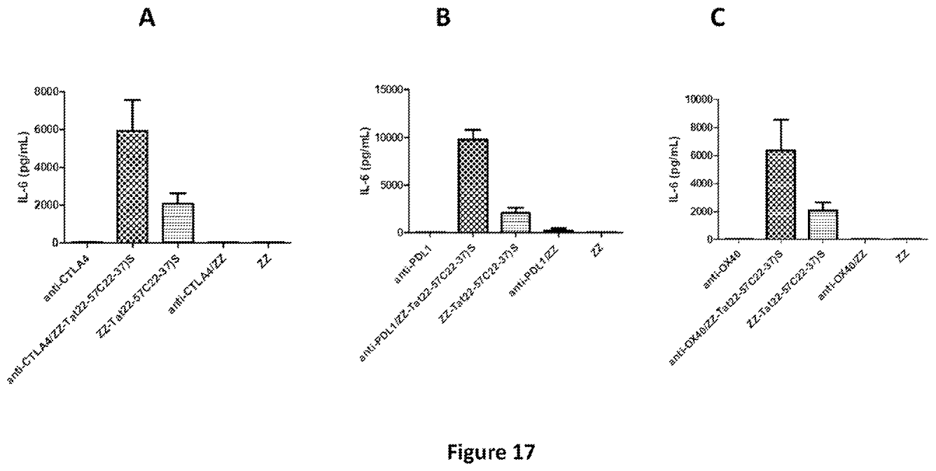

[FIG. 8A-B] shows that the proteins ZZ-DTRBD_{HEK} and ZZ-Tat_{22-57C(22-37)S} are capable of inducing the expansion of dendritic cells in mice. Three groups of mice were injected three days apart with a PBS buffer in the absence or presence of a fixed amount of ZZ-DTRBD_{HEK} (5 nmol per mouse) or ZZ-Tat_{22-57C(22-37)S} (10 nmol per mouse). 24 h after the final injection, the animals were euthanized and the spleens were removed and splenocytes labeled with a cocktail of fluorescent Ab making it possible to distinguish the cDC-CD8+ (CD11c^{high}B220-CD8⁺CD11b⁻) (A), cDC-CD11b+(CD11chighB220⁻CD8⁻CD11b⁺) (B) and the pDC (CD11c^{int}CD317⁺CD11b⁺).

FIG. 8C shows the cells analyzed by flow cytometry. The number of positive cells has been shown as a percentage relative to the total number of living splenocytes. Statistical analysis was performed using a Kruskall-Wallis test (*p<0.05).

FIG. 9

FIG. 9 shows that ZZ-DTRBD_{HEK} induces in vitro the secretion of IL-6 and of IL-12 by isolated human DCs. Isolated DCs originating from healthy human donors were incubated in the presence of a fixed amount of ZZ-DTRBD_{HEK} (1 UM). After 24 h, the supernatants are collected to evaluate the presence of the cytokines IL-6 and IL-12 by enzyme immunoassay.

FIG. 10

FIG. 10 shows that mice injected with colorectal cancer cells exhibit slowed tumor growth when they are subsequently injected with ZZ-DTRBD_{HEK} alone or in association with an adjuvant mixture. A: Sixteen C57BL/6 mice were injected subcutaneously with 0.5 M of MC38 cells. Three days later, eight mice were injected with the adjuvant mixture CpG-B 1018/Poly I:C. (30 μg for each adjuvant) and eight were not injected (controls). A second injection was carried out three days later.

B: Eighteen C57BL/6 mice were injected subcutaneously with 0.5 M of MC38 cells. Three days later, six mice were injected with ZZ-DTRBD_{HEK} in PBS buffer, six others were injected with ZZ-DTRBD_{HEK} (2 nmol per mouse) in a PBS buffer containing the adjuvant mixture CpG-B 1018/Poly I:C (30 μg for each adjuvant) and six were not injected (controls). Two additional injections were carried out three days apart. Tumor growth was monitored by measuring the tumors using calipers. Each injection is depicted by an arrow.

FIG. 11

FIG. 11 shows that mice injected with colorectal cancer cells exhibit slowed tumor growth when they are subsequently injected with ZZ-DTRBD_{HEK}, ZZ-Tat_{22-57C(22-37)S} in association with an adjuvant mixture. Thirty two C57BL/6 mice were injected subcutaneously with 0.5 M of MC38 cells. Three days later, a group of eight mice was injected with ZZ-DTRBD_{HEK} (0.96 nmol per mouse) in 50 μl of PBS containing the adjuvant mixture CpG-B 1018/PolyI:C (30 μg for each adjuvant), another group with an anti-PD-1 Ab (50 μl, 1.3 nmol per mouse), a third group with ZZ-Tat_{22-57C(22-37)S} (0.96 nmol per mouse) in 50 μl of PBS containing the adjuvant mixture CpG-B 1018/PolyI:C (30 μg for each adjuvant), and a final group of eight mice was not injected (controls). Two additional injections were carried out three days apart. Tumor growth was monitored by measuring the tumors using calipers. Each injection is depicted by an arrow.

[FIG. 11 A] Kinetics of tumor growth.

[FIG. 11 B] Kinetics of tumor growth.

[FIG. 11 C] Survival of treated animals.

FIG. 12

FIG. 12 shows that the formation of the molecular complexes ZZ-Tat_{22-57C22-37)S}, anti-DEC205/ZZ-Tat_{22-57C22-37)S} and IgG/ZZ-Tat_{22-57C22-37)S} induce, in vitro, the increase in the proportion of activated monocytes and CD4+ T lymphocytes. A fixed concentration (0.1 μM) of anti-DEC205/ZZ-Tat_{22-57C22-37)S}, IgG/ZZ-Tat_{22-57C22-37)S}, anti-DEC205/ZZ, IgG/ZZ, anti-DEC205 Ab was incubated with human PBMCs. A fixed concentration (1 μM) of ZZ-Tat_{22-57C22-37)S} or ZZ was incubated with human PBMCs. After 24 h, the cells were collected and labeled using fluorescent Ab. The proportion of activated monocytes (CD3 CD14-) in the population of living PBMCs was evaluated using the marker CD69.

FIG. 12A The proportion of activated CD4+ T lymphocytes in the living CD4+ T lymphocyte population (CD3⁻CD4⁺) was evaluated by measuring the expression of the CD69 molecule.

FIG. 12B The cells were analyzed by flow cytometry.

FIG. 13

FIG. 13 shows that molecular complexes directed against receptors expressed by dendritic cells (anti-CD74/ZZ-Tat_{22-57C22-37)S}, anti-CD209/ZZ-Tat_{22-57C22-37)S}, et anti-CD275/ZZ-Tat_{22-57C22-37)S}) induce the secretion of IL-6 by PBMCs. Human PBMCs were incubated in the presence or in the absence of free forms of the Ab, ZZ or ZZ-Tat_{22-57C(22-37)S} and also molecular complexes targeting, respectively, the molecules CD74 (A), CD209 (B) and CD275 (C). After 24 h, the supernatants were taken off and the presence of IL-6 was evaluated by enzyme immunoassay.

FIG. 14

FIG. 14 shows that a molecular complex (anti-CD335/ZZ-Tat_{22-57C22-37)S}) directed against the CD335 receptor expressed by NK and NKT cells induces an increase in the proportion of activated NK and NKT cells. PBMCs were incubated in the presence or in the absence of free forms of the Ab, ZZ or ZZ-Tat_{22-57C(22-37)S} and also the molecular complexes anti-CD335/ZZ-Tat_{22-57C22-37)S}, anti-CD335/ZZ. After 18 h, the cells were analyzed by cytometry to evaluate the expression of the co-stimulatory molecule CD69 at the surface of the NK cells (A) and NKT cells (B).

FIG. 15

FIG. 15 shows that molecular complexes directed respectively against two receptors expressed by NK and NKT cells induce an increase in the proportion of activated NKT cells. PBMCs were incubated in the presence or in the absence of free forms of the Ab, ZZ or ZZ-Tat_{22-57C(22-37)S} and also the molecular complexes anti-CD56/ZZ-Tat_{22-57C22-37)S}, anti-CD56/ZZ, anti-CD336/ZZ-Tat_{22-57C22-37)S}, or anti-CD336/ZZ. After 18 h, the cells were analyzed by cytometry to evaluate the expression of the co-stimulatory molecule CD69 at the surface of the NKT cells.

FIG. 16

FIG. 16 shows that molecular complexes directed against receptors expressed by NK cells (anti-CD56/ZZ-Tat_{22-57C22-37)S}, anti-CD335/ZZ-Tat_{22-57C22-37)S}, et anti-CD336/ZZ-Tat_{22-57C22-37)S}) induce the secretion of IL-6 by human PBMCs. PBMCs were incubated in the presence or in the absence of free forms of the Ab, ZZ or ZZ-Tat_{22-57C(22-37)S} and also molecular complexes targeting, respectively, the molecules CD56 (A), CD335 (B) and CD336 (C). After 24 h, the supernatants were taken off and the presence of IL-6 was evaluated by enzyme immunoassay.

FIG. 17

FIG. 17 shows that molecular complexes containing anti-ICP Ab (anti-CTLA-4/ZZ-Tat_{22-57C22-37)S}, anti-PD-L1/ZZ-Tat_{22-57C22-37)S}, and anti-OX40/ZZ-Tat_{22-57C22-37)S}) induce the secretion of IL-6 by human PBMCs. PBMCs were incubated in the presence or in the absence of free forms of the Ab, ZZ or ZZ-Tat_{22-57C(22-37)S} and also molecular complexes targeting, respectively, the molecules CTLA-4 (A), PD-L1 (B) and OX40 (C). After 24 h, the supernatants were taken off and the presence of IL-6 was evaluated by enzyme immunoassay.

### EXAMPLES

### Example 1: Expression of Fusion Proteins in E. coli or in HEK Cells, Purification, Biochemical Characterization, and Study of the Capacity Thereof to Bind Heparan Sulfates

### Materials and Methods

### Production of the Different Molecular Complexes

The expression of the fusion protein ZZ-DTRBD in E. coli, referred to as ZZ-DTRBD_{coli}, was previously described in the publication by Lobeck et al. (Infection and Immunity, 1998, 66, 418-423). The fusion protein ZZ-DTRBD_{coli} (SEQ ID NO: 12) is encoded by a polynucleotide having the sequence SEQ ID NO: 11. The expression of the fusion protein ZZ-Tat_{22-57C(22-37)S} in E. coli was produced according to a protocol similar to that described for the fusion ZZOVATat_{22-57S} in the publication by Knittel et al. (Vaccine, 2016, 34(27):3093-3101). The fusion protein ZZ-Tat_{22-57C(22-37)Scoli} (SEQ ID NO: 20) is encoded by a polynucleotide having the sequence SEQ ID NO: 19. For the expression in eukaryotic cells, HEK cells (2.5×10⁶ cells/ml in 250 ml 293F freestyle medium) were transfected with a pCDNA3.4 plasmid encoding ZZ-DTRBD_{HEK} (400 μg of DNA preparation resulting from maxiprep by transfection) in the presence of PEI (0.5 mg/ml). This plasmid comprises the polynucleotide having the sequence SEQ ID NO: 13 which encodes the fusion protein ZZ-DTRBD_{HEK} (SEQ ID NO: 14). The cells were subsequently incubated for 24 h at 37° C. with stirring. 250 ml of Ex-Cell medium was then added. After 4 days of incubation at 37° C. with stirring, the culture superatants were recovered, filtered under sterile conditions, and a protease inhibitor cocktail was added.

After expression of the three proteins, the supernatants were respectively diluted by ½ in 0.1% PBS-Tween then passed over an IgG sepharose column (IgG sepharose 6Fast flow #17-0969-02, Amersham) in order to purify the molecular complexes by immunoaffinity. The acidity of the fusion proteins eluted from the column was neutralized in 1 M Tris-HCl buffer, pH 8. The fusion proteins originating from the expression in E. coli, ZZ-DTRBD_{coli} and ZZ-Tat_{22-57C(22-37)S}, were subjected to a second purification cycle using a mono S 5/50 cation exchange column (GE Healthcare). The column was equilibrated with 0.05 M phosphate-citrate buffer, pH=5.5, for the purification of ZZ-DTRBD_{coli}. The column was equilibrated with 0.05 M phosphate-citrate buffer, pH=4 for the purification of ZZ-Tat_{22-57C(22-37)S}. The fusion proteins ZZ-DTRBD_{coli} and ZZ-Tat_{22-57C(22-37)S} were subsequently eluted with a linear gradient from 0 to 1 M NaCl. The proteins were finally concentrated in PBS and stored at −20° C. until use. The molecular complexes BB-DTRBD_{HEK} and BB-DTRBD_{coli} were produced according to the same protocol as used for ZZ-DTRBD_{HEK} and ZZ-DTRBD_{coli}. The fusion protein BB-DTRBD_{HEK} (SEQ ID NO: 18) is encoded by the polynucleotide sequence SEQ ID NO: 17. The fusion protein BB-DTRBD_{coli} (SEQ ID NO: 16) is encoded by the polynucleotide sequence SEQ ID NO: 15.

Analysis of the Molecular Weight and of the Degree of Homogeneity of the Molecular Complexes ZZ-DTRBD_{HEK}, ZZ-DTRBD_{coli}, BB-DTRBD_{HEK} and BB-DTRBD_{coli} by Gel Electrophoresis

The proteins ZZ-DTRBD_{HEK}, ZZ-DTRBD_{coli}, BB-DTRBD_{HEK} and BB-DTRBD_{coli} and also the molecular weight markers were deposited on SDS-PAGE 4-12% gel under denaturing conditions and then subjected to electrophoretic migration. Following the migration, the presence of protein bands was revealed using Coomassie blue staining.

### Binding of the Molecular Complexes to Heparin

In order to evaluate the binding of the molecular complexes to heparan sulfates, use was made of heparin, which is a sulfated sugar representing the heparan sulfate family.

The interaction was evaluated using an enzyme immunoassay technique. To this end, a series of microtitration plates was adsorbed beforehand with rabbit IgG (1 μg/100 μl/well in 0.1 M phosphate buffer, pH 7.2) then saturated with a buffer solution containing 0.3% bovine serum albumin (200 μl/well in 0.1 M phosphate buffer, pH 7.2). Another series of microtitration plates was saturated with a buffer solution containing 0.3% bovine serum albumin (300 μl/well in 0.1 M phosphate buffer, pH 7.2). The two series of plates were subsequently washed, and series dilutions (in 0.1 M phosphate buffer, pH 7.4, containing 0.1% bovine serum albumin) of the proteins ZZ-DTRBD_{HEK}. ZZ-DTRBD_{coli}, BB-DTRBD_{HEK} and BB-DTRBD_{coli} and ZZ-Tat_{22-57C(22-37)S} and free ZZ were deposited in the wells. After 4 hours of incubation at ambient temperature, the plates were washed and 100 μl of heparin-biotin (1 μM) were added per well. After 1 hour at ambient temperature, the plates were washed and 100 μl of streptavidin coupled to peroxidase (1/2000 dilution) was added. After 30 minutes of incubation, the plates were washed and a substrate (ABTS) was added. The staining was measured at 414 nm after 30 minutes of incubation. In order to eliminate the non-specific binding to albumin, the optical signal measured on the adsorbed plates solely with the bovine serum albumin was subtracted from the signal measured on the plates adsorbed with IgG. The heparin binding is considered to be significant when the optical density signal is greater than or equal to 50% of the signal measured when ZZ-Tat_{22-57C(22-37)S} is incubated at 100 nM, pH 7.2, on the microtitration plates.

### Results

The inventors previously constructed a fusion protein, referred to as ZZ-DTRBD, incorporating both a double ZZ domain derived from the protein A of Staphylococcus aureus and also the DTRBD domain derived from the diphtheria toxin (Lobeck et al., Infection and Immunity, 1998, 66, 418-423). ZZ can bind to the Fc region of immunoglobulins in a similar manner to the protein A. As for DTRBD, it binds to the diphtheria toxin receptor and also has a heparan sulfate binding site located in the region 453-467 (Knittel et al. J. Immunol., 2015, 194(8):3601-11; Knittel et al. Vaccine, 2016, 34(27):3093-3101). These characteristics enable ZZ-DTRBD to target different cell types bearing surface immunoglobulins and proteoglycan heparan sulfates via the interaction with the heparan sulfates.

Similarly, the inventors constructed a fusion protein, referred to as BB-DTRBD, by replacing the sequence encoding ZZ with a coding sequence referred to as BB. The BB protein corresponds to a double domain which, just like ZZ, is derived from the protein A of Staphylococcus aureus but has the particular feature of binding the Fc region and also the Fab region of immunoglobulins (Jansson 1998 FEMS Immunol. and Med. Microbiol., Léonetti et al. 1999. J. Exp. Med., 189, 1217-28).

The inventors also constructed a fusion protein, referred to as ZZ-Tat_{22-57C(22-37)S}. It contains a double ZZ domain derived from the protein A of Staphylococcus aureus and a Tat_{22-57C(22-37)S} derived from the transcriptional transactivator of HIV (WO 2011/092675, and Knittel et al. Vaccine, 2016, 34(27):3093-3101) which has a heparan sulfate binding site. These characteristics enable ZZ-Tat_{22-57C(22-37)S} to target different cell types bearing surface immunoglobulins and proteoglycan heparan sulfates via the interaction with the HS.

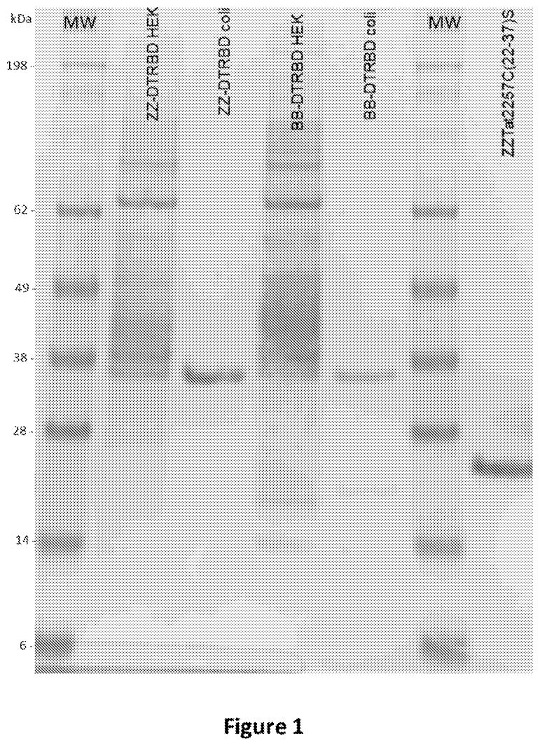

The inventors expressed ZZ-DTRBD, BB-DTRBD and ZZ-Tat_{22-57C(22-37)S} recombinantly using different expression systems. The complexes expressed in E. coli are referred to as ZZ-DTRBD_{coli}, BB-DTRBD_{coli} and ZZ-Tat_{22-57C(22-37)S} and those expressed in HEK cells are named ZZ-DTRBD_{HEK} and BB-DTRBD_{HEK}. After expression, the complexes were purified using a column containing an IgG-bearing gel. Next, the proteins ZZ-DTRBD_{coli}, BB-DTRBD_{coli} and ZZ-Tat_{22-57C(22-37)S} were subjected to ion-exchange chromatography in order to eliminate the contaminating LPS. Finally, certain characteristics of these complexes were evaluated by gel electrophoresis. As can be seen in FIG. 1, ZZ-DTRBD_{coli} and BB-DTRBD_{coli} migrate in a predominant band with a molecular weight of approximately 35 kDa which is close to the theoretical weight of the molecule (32230), indicating that this band corresponds to a monomer. Minor bands corresponding to degradation products are also present in these two molecular complexes. ZZ-DTRBD_{HEK} and BB-DTRBD_{HEK} migrate in several bands distributed substantially between 35 and 150 kDa, indicating that the expression in HEK cells leads to a heterogeneous mixture consisting of monomeric and oligomeric forms of ZZ-DTRBD. The molecular complex ZZ-Tat_{22-57C(22-37)S} migrates in a predominant band, reflecting the homogeneity of the purified protein.

The molecular weight of approximately 25 kDa is slightly greater than that calculated for the theoretical weight of the molecule (MW=19 145). However, Tat and derivatives thereof tend to migrate abnormally (Kittiworakam et al. etc), strongly suggesting that the fusion protein ZZ-Tat_{22-57C(22-37)S} expressed in E. coli and purified substantially consists of a monomer.

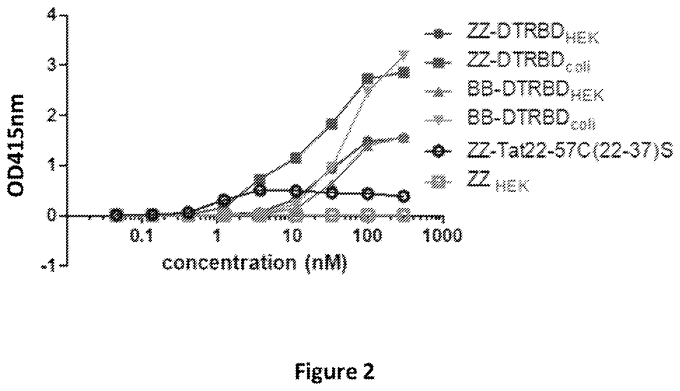

Next, the inventors studied the aptitude of free ZZ and of the five molecular complexes to bind heparin, which is a sulfated polysaccharide representing the heparan sulfate family. Enzyme immunoassay does not make it possible to observe a significant optical signal for free ZZ (FIG. 2). In contrast, an increase in the optical density on the basis of the incubated dose is measured for ZZ-DTRBD_{coli}, BB-DTRBD_{HEK}, BB-DTRBD_{coli}, ZZ-DTRBD_{HEK} and ZZ-Tat_{22-57C(22-37)S}. This data therefore indicates that the presence of the DTRBD domain or of the Tat_{22-57C(22-37)S} domain enables the molecular complexes to bind to heparin. Moreover, since the fusions ZZ-DTRBD_{HEK} and BB-DTRBD_{HEK} contain oligomers (see FIG. 1), this data indicates that the presence of oligomers does not disrupt the interaction.

### Example 2: The Fusion Protein ZZ-DTRBD_{HEK} is Capable of Binding Different Types of Cells of the Murine Immune System

### Materials and Methods

### Binding of ZZ-DTRBD to Cells of the Immune System

C57BI/6 mouse splenocytes are resuspended at 10×10⁶ cells/ml in PBS buffer, 0.5% BSA 2 mM EDTA. 100 μl of the cell suspension are deposited in a 96-round-bottomed-well plate. 100 μl of buffer, 0.5% BSA 2 mM EDTA are added per well in the absence or presence of ZZ-DTRBD_{HEK} (100 nM). The mixtures are incubated for 30 minutes at 4° C. then washed twice in PBS 0.5% BSA 2 mM EDTA. 2 μg per well of rabbit IgG are added to the cells, which are incubated for 20 minutes at 4° C. then washed twice with q.s. 200 μl of PBS 0.5% BSA 2 mM EDTA. The cells are resuspended in 50 μl of labeling buffer (PBS 0.5% BSA 2 mM EDTA) containing different mixtures of Ab from BioLegend. The mixtures are incubated for 20 minutes at 4° C. in darkness then the splenocytes are washed twice in q.s. 200 μl of PBS 0.5% BSA 2 mM EDTA. The cells are then fixed for 30 minutes at ambient temperature. To this end, 100 μl of 4% PFA buffer, then 100 μl of PBS 0.5% BSA 2 mM EDTA were added before detection on BD FACSAria™ cytometer.

Mixture 1 for the dendritic cells (duplicates): B220-FITC (#103206, 1/200), CD11 c-PE-Cy7 (#117318, 1/200), CD317-APC (#127016, 1/100), CD8a-PerCP-Cy5.5 (#100734, 1/200), CD11b-APC-Cy7 (#101226, 1/800), Donkey anti-rabbit-BV421 (#406410, 1/100), Live Dead Aqua (ThermoFischer #L34966, 1/1000).

Mixture 2 for the monocytes and T. B lymphocytes (duplicates): B220-FITC (#103206, 1/200), CD19-BV650 (#115541, 1/100), CD3-APC-Cy7 (#100222, 1/100), CD4-BV605 (#100451, 1/200), NK1.1-PE-Cy7 (#108714, 1/100), CD8a-PerCP-Cy5.5 (#100734, 1/200), CD11b-APC (#101212, 1/800), Ly6C-PE #128007, 1/800), Donkey anti-rabbit-BV421 (#406410, 1/100), Live Dead Aqua (ThermoFischer #L34966, 1/1000).

### Results

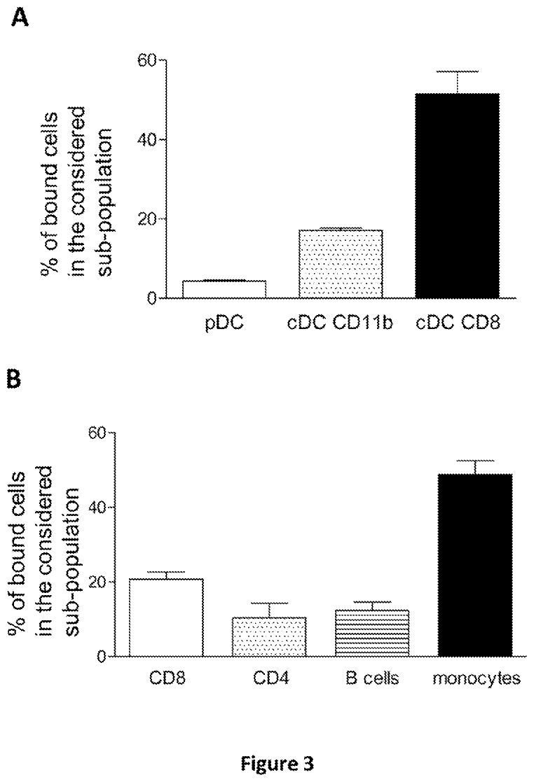

To evaluate the capacity of a molecular complex to bind cells of the immune system, ZZ-DTRBD_{HEK} was incubated in the presence of mouse splenocytes. It was thus possible to observe that this fusion protein preferentially binds to cDC-CD8+ cells which are specialized APCs (FIG. 3A). It also preferentially interacts with monocytes which may also serve as APCs (FIG. 3B). Finally, it binds to a lesser extent to lymphocytes. This data therefore indicates that the fusion protein preferentially targets APCs.

### Example 3: The Fusion Proteins ZZ-DTRBD_{HEK} and ZZ-Tat_{22-57C(22-37)S} Induce In Vitro the Secretion of IL-6 and of IL-12 by Cells of the Immune System

### Materials and Methods

C57BL/6 mouse splenocytes are resuspended at 2×10⁶ cells/ml in RPMI medium 10% FCS 1% penicillin/streptomycin. 100 μl of the cell suspension are distributed in a 96-well plate in the presence or absence of the molecular complexes (ZZ, DTRBD, ZZ-DTRBD_{HEK}, or ZZ-Tat_{22-57C(22-37)S}), incubated at a final 1 μM. After 24 h of incubation, the supernatants are collected for ELISA assay of the cytokines IL-6 and IL-12 carried out according to the manufacturer's instructions (R&D #DY406-05 and #DY419)

### Results

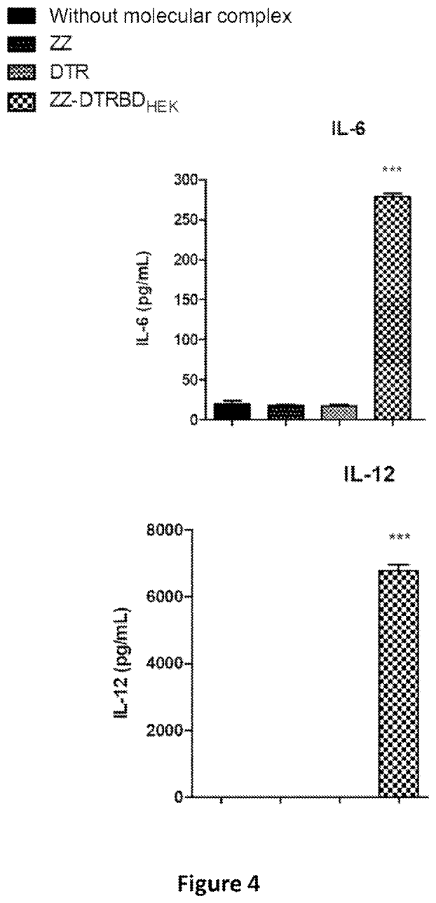

The inventors wondered if the molecular complexes could induce the activation of cells of the immune system. Since cell activation can lead to cytokine secretion, the inventors decides to evaluate in vitro the presence of two cytokines in supernatants resulting from the incubation of mouse splenocytes with molecular complexes and different control proteins. The first cytokine is IL-6, because it represents inflammatory cytokines which are important for initiating the immune response. The second is IL-12 because it is crucial for inducing cellular immune responses. The inventors found these two cytokines in increased amounts in the supernatants resulting from the incubation with ZZ-DTRBD_{HEK}, indicating that this molecular complex induces activation of the splenocytes (FIG. 4). In contrast, they did not find an increased amount of cytokines in the supernatants resulting from the incubation with free ZZ or free DTRBD, indicating that the double domain for binding to Ig and the heparan sulfate-binding domain cannot alone activate immune cells. These results therefore demonstrate that ZZ-DTRBD_{HEK} is capable of inducing activation of cells of the immune system and indicate that the association of the double Ig-binding domain and of the DTRBD HS-binding domain is absolutely required for the effect.

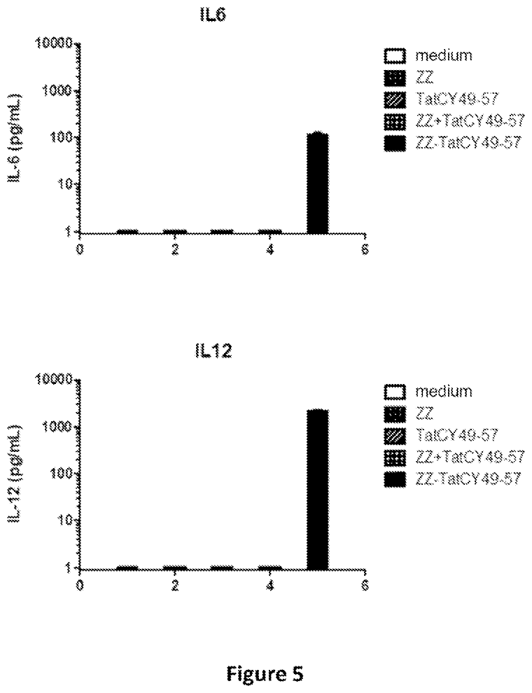

The inventors proceeded according to a principle similar to that described in the previous paragraph, in order to evaluate whether ZZ-Tat_{22-57C(22-37)S} is capable of activating cells of the immune system and if the association of ZZ and of the Tat domain involved in binding to heparan sulfates is absolutely required for the stimulatory effect. To this end, they used in particular a peptide, named Tat_{CY49-57}, which contains the basic Tat region and thus represents the interaction of Tat and derivatives thereof with heparan sulfates. They incubated mouse splenocytes with ZZ, Tat_{CY49-57}, ZZ+Tat_{CY49-57} and ZZ-Tat_{22-57C(22-37)S}, respectively. They then evaluated the presence of IL-6 and IL-12 in the supernatants. They found these two cytokines in the supernatants resulting from incubation with ZZ-Tat_{22-57C(22-37)S} but not in the supernatants resulting from incubation with ZZ, Tat_{CY49-57}, ZZ+Tat_{CY49-57} (FIG. 5). These results therefore demonstrate that ZZ-Tat_{22-57C(22-37)S} is capable of inducing the activation of cells of the immune system and that this characteristic is not shared by the free forms, ZZ and Tat_{CY49-57}, indicating that the association of the double Ig-binding domain and of the basic Tat region responsible for the HS binding is absolutely required for the effect.

### Example 4: A Molecular Complex i) Remains Capable of Triggering Secretion of IL-6 and IL-12 by Cells of the Immune System when the ZZ Domain Thereof is Substituted by BB, ii) has Increased Stimulatory Capacities when it Consists of Oligomeric Forms

### Materials and Method

C57BL/6 mouse splenocytes are resuspended at 2×10⁶ cells/ml in RPMI medium 10% FCS 1% penicillin/streptomycin. 100 μl of the cell suspension are distributed in a 96-well plate in the presence or absence of different molecular complexes (ZZ-DTRBD_{HEK}, BB-DTRBD_{HEK}, ZZ-DTRBD_{coli} and BB-DTRBD_{coli}) incubated at a final 1 μM. After 24 h of incubation, the supernatants are collected for ELISA assay of the cytokines IL-6 and IL-12 carried out following the manufacturer's instructions (R&D #DY406-05 and #DY419).

### Results

The work described in example 3 was carried out with two molecular complexes containing the ZZ double domain. This double domain, which binds the Fc of Ab, can thus target the Ab located at the surface of the APCs. The inventors then wondered if molecular complexes which can jointly target the Fc region and the Fab region of the Ig might also be capable of activating cells of the immune system. To this end, they focused on the BB double domain derived from the protein A of Staphylococcus aureus, which has the particular feature of being able to bind the Fc region of the Ab in the same way as Z, but also the Fab region of the Ab, unlike ZZ (Jansson B. et al., Fems Immunol. Med. Microbiol. 1998, 20:69-78). They prepared the complexes BB-DTRBD_{HEK} and BB-DTRBD_{coli} which are described in example 1, and compared them to ZZ-DTRBD_{HEK} and ZZ-DTRBD_{coli} for the capacity to induce secretion of the cytokines IL-6 and IL-12 in vitro when they are incubated with murine splenocytes.

Since the inventors noted that ZZ-DTRBD and BB-DTRBD are in oligomeric form following their expression in HEK cells (see FIG. 1) whereas they are monomeric after expression in E. coli, this work also made it possible to evaluate if the state of oligomerization could affect the capacity to induce the immune system.

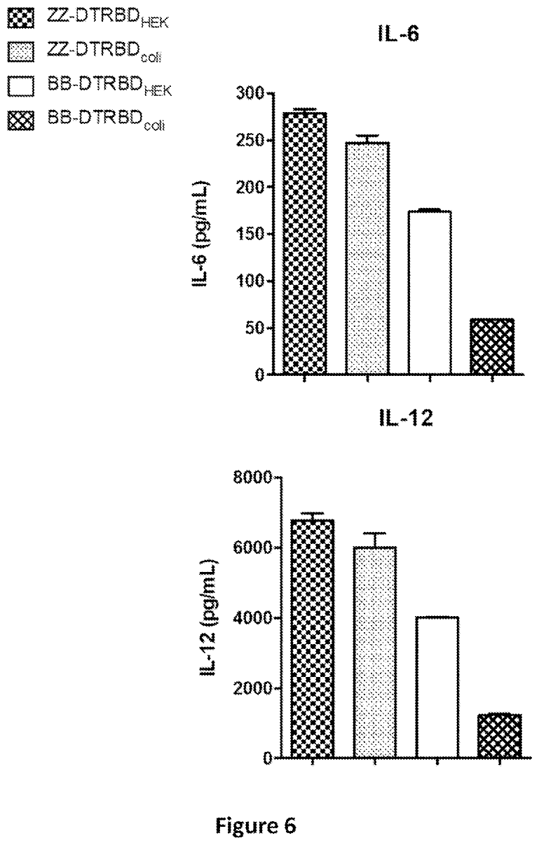

The inventors observed that the supernatants resulting from incubation with ZZ-DTRBD_{HEK} contained greater amounts of cytokines than those resulting from incubation with BB-DTRBD_{HEK} (FIG. 6). Similarly, the supernatants resulting from incubation with ZZ-DTRBD_{coli} contained greater amounts of cytokines than those resulting from incubation with BB-DTRBD_{coli}. These results demonstrate that a molecular complex containing BB remains capable of inducing the secretion of cytokines in vitro, but to a lesser extent than a molecular complex containing ZZ. This data therefore indicates that the activation effect can be mediated by molecular complexes containing domains capable of binding different sites on Ab.

The comparison of the contents of cytokines in the supernatants based on the cell type used (i.e. HEK vs. E. coli) for producing the molecular complexes made it possible to demonstrate the difference in stimulatory efficacy depending on the type of production. Indeed, in the supernatants resulting from incubation with complexes originating from HEK cells (ZZ-DTRBD_{HEK}, BB-DTRBD_{HEK}) IL-6 and IL-12 are present in a greater amount than those resulting from incubation with complexes resulting from expression in E. coli (ZZ-DTRBD_{coli} and BB-DTRBD_{coli}) (FIG. 6). Since the proteins resulting from expression in HEK cells are mainly oligomeric (see FIG. 1), the inventors deduced from this that the immune system is more effectively induced when the molecular complexes have a high degree of oligomerization.

### Example 5: Isolated Dendritic Cells Secrete IL-6 and IL-12 when they are Incubated in the Presence of ZZ-DTRBD_{HEK}

### Materials and Methods

A C57BL/6J mouse is euthanized, then its spleen is recovered in RPMI medium 10% FCS 1% penicillin/streptomycin. The spleen is perfused with 3 ml collagenase D at 2 mg/ml in HBSS 0.5% BSA then incubated for 30 minutes at 37° C. The splenocytes are recovered and magnetic sorting for the dendritic cells (DC) is carried out according to the manufacturer's instructions (Miltenyi Biotec #130-100-875). The DC are centrifuged for 5 minutes at 4° C. at 390×g then resuspended at 2×10⁶ cells/ml in RPMI medium 10% FCS 1% penicillin/streptomycin. 100 μl of the cell suspension (200 000 cells) are placed in a 96-flat-bottomed-well plate and 100 μl of RPMI medium 10% FCS 1% penicillin/streptomycin is added in the absence or presence of ZZ-DTRBD_{HEK} (final 0.6 μM). The cells are incubated for 24 h at 37° C., then the supernatants are collected for ELISA assay of the cytokines IL-6 and IL-12 according to the manufacturer's instructions (R&D #DY406-05 and #DY419).

### Results

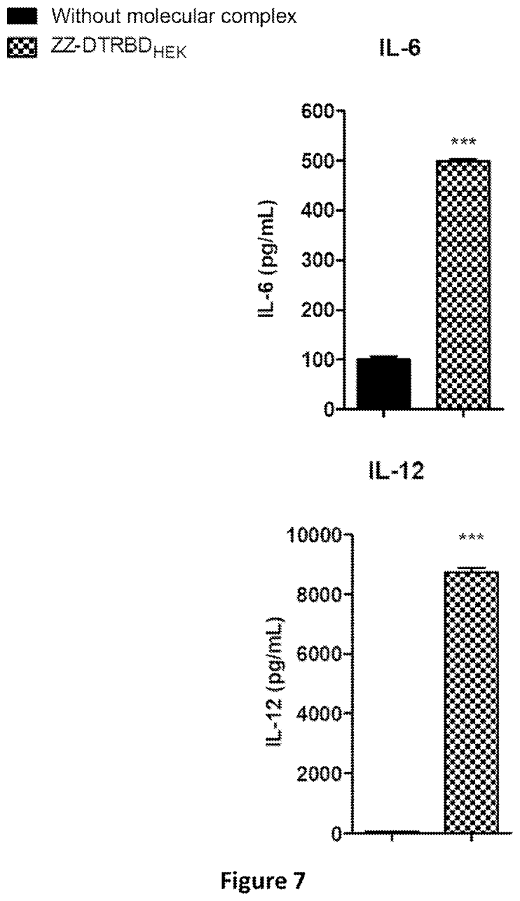

The establishing of immune defense mechanisms depends on the collaboration between different cell partners. Among these, dendritic cells (DC) represent APCs playing a central role. This is because they contribute to activating other cell types, via direct interactions or via cytokines which they secrete. In order to evaluate if ZZ-DTRBD induces cytokine secretion by DCs. DCs were purified from mouse splenocytes C57BI/6. Next, these DCs were incubated for 24 h in the presence or absence of ZZ-DTRBD, then the supernatants were taken off in order to assay the presence of IL-6 and IL-12 (FIG. 7). The amount of IL-6 present in the supernatants resulting from incubation with ZZ-DTRBD_{HEK} is approximately 5 times greater than that found in the superatants resulting from incubation without a molecular complex. A large amount of IL-12 (approximately 8700 pg/ml) is also detected in the supernatants resulting from incubation with ZZ-DTRBD_{HEK} but virtually none in those resulting from incubation without a molecular complex. This data therefore indicates that ZZ-DTRBD causes the secretion of IL-6 and of IL-12 by murine dendritic cells and could thus contribute to establishing immune defense mechanisms.

### Example 6: The Proteins ZZ-DTRBD and ZZ-Tat_{22-57C(22-37)S} are Capable of Inducing Dendritic Cells in Mice

### Materials and Methods

Three groups of eight C57BL/6 mice were injected three times, at three day intervals, with 100 μl PBS in the absence or presence of ZZ-DTRBD_{HEK} (5 nmol per mouse) or ZZ-Tat_{22-57C(22-37)S} (10 nmol per mouse), respectively. 24 hours after the final injection, the animals were euthanized, the spleens were collected to recover the splenocytes. The cells were resuspended in 50 μl of labeling buffer (PBS 0.5% BSA 2 mM EDTA) containing different mixtures of Ab. The cells were then incubated for 20 minutes at 4° C. in darkness then washed twice in PBS 0.5% BSA 2 mM EDTA. The cells were then fixed for 20 minutes at 4° C. in 100 μl of 4% PFA buffer then washed in PBS 0.5% BSA 2 mM EDTA. They were finally resuspended in 200 μl of PBS 0.5% BSA 2 mM EDTA then analyzing using a BD FACSAria™ flow cytometer. The cells were analyzed by flow cytometry. The cDC-CD8+ are identified as CD11c^{high}B220⁻CD8⁻CD11b⁻, the cDC-CD11b+ are identified as CD11c^{high}B220⁻CD8⁻CD11b⁻ and the pDC are identified as CD11c^{int}CD317⁺CD11b⁺.

The mixtures of Ab used for the phenotype analysis of the dendritic cells are as follows: B220-FITC (#103206, 1/200), CD11c-PE-Cy7 (#117318, 1/200), CD317-APC (#127016, 1/100), CD11b-APC-Cy7 (#101226, 1/800), CD8a-PerCP-Cy5.5 (#100734, 1/200), Live Dead Violet (ThermoFischer #L34964, 1/1000).

### Results

Since the molecular complexes bind preferentially to APCs in vitro and induce cytokine secretion, the inventors wondered if such complexes might induce the expansion of APCs in mice. In order to evaluate this aspect, the inventors injected three groups of 8 C57BI/6 mice with a PBS solution in the presence or absence of ZZ-DTRBD_{HEK} or ZZ-Tat_{22-57C(22-37)S}. They then euthanized the animals and sampled their spleen in order to evaluate the frequency of the different sub-populations of dendritic cells in the splenocytes. As can be seen in FIG. 8, the three murine DC populations (i.e. DC-CD8+, DC-CD11b+ and pDC) are present in the spleens of the group of mice injected solely with PBS. However, the frequency of these three cell types is significantly increased in the groups of animals injected with ZZ-DTRBD_{HEK} or ZZ-Tat_{22-57C(22-37)S}. This data therefore indicates that the molecular complexes are capable of inducing the expansion of the dendritic cells in vivo. Since these cells are central to initiating the immune response, these results strongly suggest that the complexes can thus promote the induction of immune response mechanisms.

### Example 7: ZZ-DTRBD_{HEK} Induces the Secretion of IL-6 and of IL-12 by Human Dendritic Cells In Vitro

### Materials and Methods

### Isolation of Human Dendritic Cells and Incubation for Studying the Secretion of IL-6 and IL-12

A leukocyte-platelet layer bag is diluted to ½ in AIM V medium, then incubated overnight at ambient temperature with stirring. 15 ml of histopaque medium is then added to 4 leucosep tubes, to which are added 4×25 ml of diluted blood. The tubes are centrifuged unrestrictedly for 15 minutes at ambient temperature at 1000×g. The rings of peripheral blood mononuclear cells (PBMCs) are recovered and washed in PBS 2 mM EDTA without calcium or magnesium. The PBMCs are centrifuged at 150×g for 10 minutes at ambient temperature then a red blood cell-lyzing buffer (8.3 mg/ml NH4CI, 0.84 mg/ml NaHCO₃, 0.1 mM EDTA) is added and incubated for 10 minutes at 4° C. 40 ml of PBS are added, then the cells are centrifuged for 10 minutes at ambient temperature and at 150×g. The DCs are then sorted according to the manufacturer's instructions (Miltenyi Biotec #130-091-379). The DC are centrifuged for 5 minutes at 4° C. at 390×g then resuspended at 2×10⁶ cells/ml in RPMI medium 10% FCS 1% penicillin/streptomycin. 100 μl of the sorted DCs (200 000 cells) are placed in a 96-well plate and 100 μl of RPMI medium 10% FCS 1% penicillin/streptomycin is added in the absence or presence of ZZ-DTRBD_{HEK} (final 1 μM). The cells are incubated for 24 h at 37° C., then the supernatants are collected for ELISA assay of the cytokines IL-6 and IL-12 according to the manufacturer's instructions (R&D #DY206-05 and #DY1270-05).

### Results

To evaluate if ZZ-DTRBD_{HEK} can induce in vitro the secretion of IL-6 and IL-12 by DCs originating from healthy human donors, after 24 h of incubation the presence of these two cytokines was evaluated in the culture supernatants of these cells. As can be seen in FIG. 9, IL-6 is found in an approximately 10-times greater amount in the supernatants resulting from the incubation in the presence of ZZ-DTRBD_{HEK} than in the supernatants resulting from the incubation in the absence of the fusion protein. IL-12 is present in a large amount (2400 pg/ml) in the supernatants resulting from the incubation of the DCs in the presence of ZZ-DTRBD_{HEK} and in a very low amount (8 pg/ml) in those resulting from the incubation in the absence of the molecular complex. All this data thus indicates that ZZ-DTRBD induces an increase in the secretion of IL-6 and IL-12 by human DCs. ZZ-DTRBD could thus contribute to establishing immune defense mechanisms in humans which are linked to the secretion of these cytokines.

### Example 8: ZZ-DTRBD and ZZ-Tat_{22-57C(22-37)S} Slow the Growth Progression of a Colorectal Tumor

### Materials and Methods

### Study of the Effect of the Adjuvant Mixture CpG/PolyI:C on the Tumor Growth of a Murine Colorectal Cancer Cell Line

Two groups of eight C57BL/6 mice were injected subcutaneously in the paw with 0.5 M of MC38 cells. The mice were then not injected (controls) or injected with the adjuvant mixture CpG-B 1018/Poly I:C (30 μg for each adjuvant) three and six days later. Tumor growth was monitored by measuring the tumors using calipers. Upon reaching the cessation criterion, they were euthanized.

### Study of the Effect of the Molecular Complex ZZ-DTRBD_{HEK}, Injected in the Absence or Presence of Adjuvant, on the Tumor Growth of a Murine Colorectal Cancer Cell Line

Three groups of six C57BL/6 mice were injected subcutaneously in the paw with 0.5 M of MC38 cells. Three, six and nine days later, the mice are either not injected (controls) or are injected with ZZ-DTRBD_{HEK} (2 nmol per mouse) in the absence or presence of CpG-B 1018+Poly I:C. (30 μg for each adjuvant per mouse). The mice are monitored individually for 15 days (tumor measurements using calipers) before euthanasia.

Comparison of the Effect of an Anti-PD-1 Ab and of the Molecular Complexes ZZ-DTRBD_{HEK} Et ZZ-Tat_{22-57C(22-37)S} on the Tumor Growth of a Murine Colorectal Cancer Cell Line

Four groups of eight C57BL/6 mice were injected subcutaneously in the paw with 0.5 M of MC38 cells. Three, six and nine days later, the mice are either not injected (controls) or are injected with the anti-PD-1 antibody (Euromedex #BE0146-100MG, clone RMP1-14), ZZ-DTRBD_{HEK} or ZZ-Tat_{22-57C(22-37)S} (0.96 nmol per mouse) in the presence of CpG-B 1018+Poly I:C (30 μg for each adjuvant per mouse). The mice are monitored individually over time (tumor measurements using calipers). Upon reaching the cessation criterion, they are euthanized.

### Results

In order to evaluate if the molecular complexes can have an impact on the growth of a tumor, studies were carried out in a syngeneic model of murine cancer. This model is based on a colon tumor cell line, referred to as MC38, and C57BI/6 mice. The induction of the cancer is caused by injecting 500 000 MC38 cells per mouse.

Firstly, the impact on tumor growth of the adjuvant mixture CpG1018/polyI:C was evaluated. As can be seen in FIG. 10A, compared to the untreated (control) group, tumor growth is virtually unaffected in the group of mice injected with the CpG1018/polyI:C mixture. This data therefore indicates that the adjuvant mixture alone cannot slow the growth of this colorectal tumor.

Secondly, the impact on tumor growth of ZZ-DTRBD_{HEK} injected alone or in the presence of the adjuvant mixture CpG1018/polyI:C was evaluated. As can be seen in FIG. 10B, compared to the untreated (control) group, tumor growth is slowed on day 8 and on day 10 in the group injected with ZZ-DTRBD_{HEK} without adjuvant. The effect is even greater in the group treated with ZZ-DTRBD_{HEK} and the CpG1018/polyI:C adjuvant mixture. Indeed, in this group, tumor growth is slowed over the whole period of observation of the animals (day 6 to day 15). All of this data therefore indicates that ZZ-DTRBD_{HEK} has an impact on the growth of the MC38 cell line and that this effect is amplified when it is mixed with the adjuvant CpG1018/polyI:C.

In another series of experiments, the effect on the growth of the MC38 tumor in C57BI/6 mice was compared for the following treatments: ZZ DTRBD_{HEK}/CpG1018/polyI:C, ZZ-Tat_{22-57C(22-37)S}/CpG1018/polyI:C, anti-PD-1 Ab. As can be seen in FIGS. 11A and 11B, the treatment with the anti-PD-1 Ab does not have a significant impact on tumor growth compared to the control group. In contrast, the mice injected respectively with ZZ-DTRBD_{HEK}/CPG1018/polyI:C and ZZ-Tat_{22-57C(22-37)S}/CpG1018/polyI:C exhibit decreased tumor growth. This slowing of tumor growth leads to an increased survival of the animals in these two groups (FIG. 11C).

### Example 9: CD4+ Activated Monocytes and Lymphocytes can be Induced by Molecular Complexes Targeting, Respectively, the Surface Molecule DEC205 and Fc_{gamma} Receptors

### Materials and Methods

In order to prepare a molecular complex targeting the DEC205 molecule, an anti-DEC205 Ab was used (BioLegend; clone NLDC-145, ref BLE138202). It was incubated in the absence or presence of ZZ-Tat_{22-57C22-37)S} at a fixed concentration (0.2 UM for each molecule) for 24 hours in RPMI medium without FCS. The capacity of the ZZ region to bind the Fc region of the anti-DEC205 Ab made it possible to form a non-covalent molecular complex, referred to as anti-DEC205/ZZ-Tat_{22-57C22-37)S}. The same protocol was used to form a complex between the ZZ molecule and the anti-DEC205 Ab, referred to as anti-DEC205/ZZ.

In order to prepare a molecular complex targeting the FC_{gamma} receptors, use was made of a non-specific human polyclonal Ab. This Ab was incubated with ZZ-Tat_{22-57C22-37)S} or ZZ, following an identical protocol to that used for the anti-DEC205 Ab. It was thus possible to form a non-covalent molecular complex, referred to as IgG/ZZ-Tat_{22-57C22-37)S}, and an IgG/ZZ complex.

Human PBMCs prepared as described in example 7 were then incubated in the absence or presence of the following compounds: anti-DEC205/ZZ-Tat_{22-57C22-37)S}, IgG/ZZ-Tat_{22-57C22-37)S}, anti-DEC205/ZZ, IgG/ZZ ZZ-DTRBD_{HEK}, and the anti-DEC205 Ab and free IgG (final concentration of 0.1 UM for each compound). In these experiments, PBMCs were also incubated with ZZ-Tat_{22-57C22-37)S} and free ZZ proteins at a concentration of 1 M. After 24 h, the cells were collected, labeled using fluorescent Ab making it possible to identify the monocytes (CD14, anti-CD14-BV605, Biolegend), the CD4+ T lymphocytes (CD4, anti-CD4-PerCP-Cy5.5, Biolegend) and the CD69 molecule (anti-CD69-BV785, Biolegend). After 30 minutes of incubation, the PBMCs were fixed using a solution containing 4% paraformaldehyde then analyzed by flow cytometry.

### Results

The previous examples show that molecular complexes targeting Ab can effectively induce certain immune response mechanisms and effectively slow tumor growth. The Ab located at the surface of the APCs are the molecules targeted by these complexes. However, the APCs express a large number of other molecules which may also represent targets for molecular complexes according to the invention. The inventors thus wondered if molecular complexes targeting HS and receptors other than the immunoglobulins located at the APC surface might also activate these cells. In order to evaluate this aspect, they chose to evaluate two types of receptors. The first is the protein DEC205, which is a lectin selectively expressed in humans by monocytes and certain populations of dendritic cells (Kato M. et al. 2006, Int. Immunol., 18:857-869). The second is the FC_{gamma} receptor, most forms of which are expressed by monocytes and certain populations of dendritic cells.

The inventors then prepared two molecular complexes which can respectively target these receptors. To target the DEC205 protein, they used a monoclonal Ab specific to this receptor, which they complexed to the fusion protein ZZ-Tat_{22-57C22-37)S}. They referred to this molecular complex as anti-DEC205/ZZ-Tat_{22-57C22-37)S}. In order to target the Fc receptors, they used a non-specific human polyclonal IgG Ab which can interact with these receptors via its Fc domain. They formed a molecular complex between this IgG and ZZ-Tat_{22-57C22-37)S}, referred to as IgG/ZZ-Tat_{22-57C22-37)S}. These two abovementioned molecular complexes thus have the capacity to bind APC receptors and heparan sulfates via the Tat_{22-57C22-37)S} domain of ZZ-Tat_{22-57C22-37)S}. In these molecular complexes, the two proteins are non-covalently associated via their respective Fc and ZZ domains. Therefore, ZZ can no longer target the immunoglobulins located at the surface of the APCs, because it is already interacting with the Ab. The inventors also prepared two complexes, respectively referred to as anti-DEC205/ZZ and IgG/ZZ, free of the Tat_{22-57C22-37)S} region for binding to heparan sulfates, in order to use them as a control in subsequent activation experiments.

Since the DEC205 molecule and the Fc receptors are expressed by monocytes, the inventors then wondered if the molecular complexes might enable the activation of this sub-population of APCs. To this end, they incubated PBMCs in the absence or presence of a fixed concentration (0.1 μM) of anti-DEC205/ZZ-Tat_{22-57C22-37)S}, IgG/ZZ-Tat_{22-57C22-37)S}, anti-DEC205/ZZ, IgG/ZZ, anti-DEC205, IgG, respectively. They also incubated the ZZ-Tat_{22-57C22-37)S} and free ZZ proteins at a concentration of 1 μM in order to evaluate the effect of ZZ-Tat_{22-57C22-37)S} at an identical concentration to that which was found to be activating in examples 3, 4 and 5. After 24 h, they evaluated the proportion of activated monocytes and CD4+ T lymphocytes. As can be seen in FIG. 12A, ZZ does not modify the proportion of monocytes activated in the PBMCs, while it is significantly increased by ZZ-Tat_{22-57C22-37)S}, indicating that this molecular complex makes it possible to activate human APCs. The proportion of cells activated is reduced with the anti-DEC205 Ab, free or included in the anti-DEC205/ZZ complex. In contrast, the proportion of activated monocytes is increased in the presence of the anti-DEC205/ZZ-Tat_{22-57C22-37)S} complex. Similar behavior is observed when the CD4+ T lymphocytes are considered (FIG. 12B). This data therefore indicates that an Ab targeting APCs and not having the capacity to activate them may become capable of inducing them when it is included in a molecular complex which also makes it possible to target HS.

The analysis of the state of activation of the monocytes after incubation with free human IgG shows that this Ab makes it possible to increase the proportion of activated monocytes. The IgG/ZZ does not enable a significant increase in the proportion of activated cells. In contrast, the number of activated monocytes is more than two times greater when the PBMCs are incubated with the molecular complex IgG/ZZ-Tat_{22-57C22-37)S}. Similar behavior is observed when the CD4+ T lymphocytes are considered (FIG. 12B). This data therefore indicates that an Ab targeting FC_{gamma} receptors at the surface of APCs is capable of activating them but that the activation effect is increased when the Ab is included in a molecular complex making it possible to also target HS.

Interestingly, these results show a joint increase in the proportion of activated monocytes and T lymphocytes, indicating that the molecular complexes induce several cell actors which have a central role in immune defense mechanisms.

### Example 10: Molecular Complexes Targeting Dendritic Cells Induce In Vitro the Secretion of IL-6 by Cells of the Immune System

### Materials and Methods

In order to prepare molecular complexes targeting DCs, three antibodies specific to molecules expressed by DCs were used. The first Ab (BD reference 555538), referred to as anti-CD74 Ab, targets the CD74 molecule. The second Ab (BD reference 551186), referred to as anti-CD209 Ab, targets the CD209 molecule. The third Ab (BD reference 552501), referred to as anti-CD275 Ab, targets the CD275 molecule.

Each of these three Ab (30 nM for each molecule) was incubated in the absence or presence of ZZ-Tat_{22-57C22-37)S} or ZZ (5 nM for each molecule), for 24 hours at 4° C. in RPMI medium 5% human AB serum. The capacity of the ZZ region to bind the Fc region of these Ab made it possible to form different non-covalent molecular complexes, referred to as anti-CD74/ZZ-Tat_{22-57C22-37)S}, anti-CD209/ZZ-Tat_{22-57C22-37)S}, anti-CD275/ZZ-Tat_{22-57C22-37)S}, anti-CD74/ZZ, anti-CD209/ZZ, and anti-CD275/ZZ, respectively.

Human PBMCs are resuspended at 5×10⁶ cells/ml in RPMI medium 5% human AB serum. 100 μl of the cell suspension was distributed in a 96-well plate in the presence or absence of free Ab, ZZ or ZZ-Tat_{22-57C(22-37)S} and also the molecular complexes. After 24 h of incubation, the supernatants are collected for ELISA assay of the cytokine IL-6 carried out according to the manufacturer's instructions (R&D #DY406-05)

### Results

The inventors wondered if molecular complexes targeting three molecules expressed by DCs could induce the activation of cells of the immune system. Since cell activation can lead to cytokine secretion, the inventors decided to evaluate in vitro the presence of IL-6. They found this cytokine in the supernatants resulting from incubation with ZZ-Tat_{22-57C22-37)S} but not in the supernatants resulting from incubation with ZZ. They did not find any in the supernatants resulting from incubation of PBMCs with the free anti-CD74 Ab (FIG. 13A), indicating that this Ab cannot induce activation when it is in the free form. The inventors observed that the secretion of IL-6 is increased when the PBMCs are incubated with the anti-CD74/ZZ complex, indicating that the ZZ double domain enables activation mediated by this Ab (FIG. 13A). However, the secretion of this cytokine is even stronger with anti-CD74/ZZ-Tat_{22-57C22-37)S}, indicating that the addition of the Tat region making it possible to bind HSPGs makes it possible to increase cell activation mediated by this complex.

The inventors observed that free anti-CD209 and anti-CD275 Ab are capable of inducing the secretion of IL-6 when they are incubated with PBMCs (FIGS. 13B and 13C). When these two Ab are respectively complexed to ZZ (anti-CD209/ZZ and anti-CD275/ZZ), the secretion of this cytokine is also observed. However, this secretion is increased when these two Ab are complexed to ZZ-Tat_{22-57C22-37)S}, indicating that the activating power can be increased when the molecular targeting complexes are capable of binding HSPGs.

### Example 11: A Molecular Complex Directed Against the CD335 Receptor Expressed by NK and NKT Cells Induces In Vitro an Increase in the Proportion of Activated NK and NKT Cells

### Materials and Methods

In order to prepare molecular complexes targeting the CD335 molecule at the surface of NK and NKT cells, an Ab (Origene AM31284AF-N), referred to as anti-CD335 Ab, was used. This Ab was incubated at 30 nM in the absence or presence of ZZ-Tat_{22-57C22-37)S} or ZZ (5 nM for each molecule), for 5 hours at 37° C. in RPMI medium, 5% human AB serum. The capacity of the ZZ region to bind the Fc region of these Ab made it possible to form two molecular complexes, referred to as anti-CD335/ZZ-Tat_{22-57C22-37)S} and anti-CD335/ZZ, respectively.

Human PBMCs are resuspended at 5×10⁶ cells/ml in RPMI medium 5% human AB serum. 100 μl of the cell suspension was distributed in a 96-well plate in the presence or absence of the anti-CD335 Ab, ZZ or ZZ-Tat_{22-57C(22-37)S} and also the molecular complexes. After 18 h, the cells were collected, labeled using fluorescent Ab making it possible to identify the NK cells (CD56+, biolegend reference 362510 dilution to 1/100), NKT cells (CD56+ biolegend reference 362510; CD3+ cells; Miltenyi ref 130-113-136 dilution to 1/200), and the CD69 molecule (biolegend reference 310932, dilution to 1/100). After 30 minutes of incubation, the PBMCs were fixed using a solution containing 4% paraformaldehyde then analyzed by flow cytometry.

### Results

The inventors wondered if molecular complexes targeting the CD335 molecule expressed at the surface of NK and NKT cells could induce the activation of these two cell types. To evaluate this, they used the anti-CD335 Ab in isolated form or included in a molecular complex. They incubated PBMCs in the absence or presence of the different mixtures. Since NK and NKT cells have a crucial role in immune defense mechanisms, after 18 h of incubation they evaluated the proportion of activated NK and NKT cells by monitoring the expression of the co-stimulatory molecule CD69.

As can be seen in FIG. 14, ZZ, the isolated anti-CD335 Ab and the anti-CD335/ZZ complex do not increase the proportion of NK (A) or NKT (B) cells expressing CD69 in PBMCs. Different behavior is observed with free ZZ-Tat_{22-57C22-37)S}. Indeed, this molecular complex does not increase the proportion of NKT cells expressing CD69, but it does increase the percentage of NK cells expressing this marker, indicating that the association of ZZ and of the HS ligand in the ZZ-Tat_{22-57C(22-37)S} complex makes it possible to increase the activation of NK cells. The molecular complex anti-CD335/ZZ-Tat_{22-57C(22-37)S} has an impact on the activation of the two cell sub-populations. Indeed, it increases the proportion of NK and NKT cells expressing CD69. All this data therefore indicates that the anti-CD335 Ab does not have the capacity to activate the two cell types, but may become capable of inducing them when it is included in a molecular complex which also makes it possible to target HS.

### Example 12: Molecular Complexes Respectively Directed Against the CD56 (A) and CD336 (B) Molecules Expressed by NK and NKT Cells Induce In Vitro an Increase in the Proportion of Activated NKT Cells

### Materials and Methods

In order to prepare molecular complexes targeting the CD56 and CD336 molecules at the surface of the NK and NKT cells, the Ab anti-CD56 (Biolegend reference 304622) which targets the CD56 molecule, and the Ab anti-CD336 (Origene AM50346PU-N) which targets the CD336 molecule were used.

These Ab were incubated at 30 nM in the absence or presence of ZZ-Tat_{22-57C22-37)S} or ZZ (5 nM for each molecule), for 5 hours at 37° C. in RPMI medium, 5% human AB serum. The capacity of the ZZ region to bind the Fc region of these Ab made it possible to form four molecular complexes, referred to as anti-CD56/ZZ-Tat_{22-57C22-37)S}, anti-CD56/ZZ, anti-CD336/ZZ-Tat_{22-57C22-37)S}, and anti-CD336/ZZ, respectively.

Human PBMCs are resuspended at 5×10⁶ cells/ml in RPMI medium 5% human AB serum. 100 μl of the cell suspension was distributed in a 96-well plate in the presence or absence of the isolated forms of the anti-CD56 Ab, anti-CD336 Ab, anti-CD336 Ab. ZZ or ZZ-Tat_{22-57C(22-37)S} and also the non-covalent molecular complexes. After 18 h, the cells were collected, labeled using fluorescent Ab described in example 11 which make it possible to identify the NKT cells (CD56+CD3+), and the CD69 molecule. After 30 minutes of incubation, the PBMCs were fixed using a solution containing 4% paraformaldehyde then analyzed by flow cytometry.

### Results

The inventors wondered if molecular complexes targeting the expressed molecules CD56 and CD336 could induce the activation of NKT cells. To evaluate this, they used the anti-CD56 and anti-CD336 Ab in isolated form or included in a molecular complex. They incubated PBMCs in the absence or presence of the different mixtures. After 18 h of incubation, they evaluated the proportion of activated NKTs by monitoring the expression of the co-stimulatory molecule CD69.

As can be seen in FIG. 15, ZZ, ZZ-Tat_{22-57C22-37)S}, the anti-CD56 Ab, the isolated anti-CD336 Ab and the anti-CD336/ZZ complex do not increase the proportion of NKT cells expressing CD69 in PBMCs. Different behavior is observed with the molecular complexes anti-CD56/ZZ-Tat_{22-57C22-37)S} (FIG. 15A), anti-CD336/ZZ-Tat_{22-57C22-37)S} (FIG. 15B). Indeed, an increase in the proportion of NKT cells expressing CD69 is observed when these two molecular complexes are incubated with PBMCs. All this data therefore indicates that the isolated anti-CD56 Ab and anti-CD336 Ab do not have the capacity to activate NKT cells, but may become capable of inducing them when they are included in a molecular complex which also targets HS.

### Example 13: Molecular Complexes Targeting NK and NKT Cells Induce In Vitro the Secretion of IL-6 by Cells of the Immune System

### Materials and Methods

In order to prepare molecular complexes targeting NK and NKT cells, the three antibodies described in examples 10 and 11 were used. Each of these three Ab (30 nM for each molecule) was incubated in the absence or presence of ZZ-Tat_{22-57C22-37)S} of ZZ (5 nM for each molecule), for 24 hours in RPMI medium 5% human AB serum. The capacity of the ZZ region to bind the Fc region of these Ab made it possible to form different non-covalent molecular complexes, referred to as anti-CD56/ZZ-Tat_{22-57C22-37)S}, anti-CD335/ZZ-Tat_{22-57C22-37)S}, anti-CD336/ZZ-Tat_{22-57C22-37)S}, anti-CD56/ZZ, anti-CD335/ZZ, and anti-CD336/ZZ, respectively.

Human PBMCs are resuspended at 5×10⁶ cells/ml in RPMI medium 5% human AB serum. 100 μl of the cell suspension are distributed in a 96-well plate in the presence or absence of free Ab, ZZ or ZZ-Tat_{22-57C(22-37)S} and also the molecular complexes. After 24 h of incubation, the superatants are collected for ELISA assay of the cytokine IL-6 carried out according to the manufacturer's instructions (R&D #DY406-05).

### Results

The inventors wondered if molecular complexes targeting NK and NKT cells could induce the activation of cells of the immune system. Since cell activation can lead to cytokine secretion, the inventors decided to evaluate in vitro the presence of IL-6. They found this cytokine in the supernatants resulting from incubation with ZZ-Tat_{22-57C22-37)S} but not in the supernatants resulting from incubation with ZZ. They did not find any IL-6 in the supernatants resulting from incubation of PBMCs with the free anti-CD56 and anti-CD335 Ab (FIGS. 16A and 16B), indicating that these two proteins cannot induce activation when they are in the free form. In contrast, they detected the cytokine in the superatants resulting from the incubation with the anti-CD336 antibody (FIG. 16C), indicating that, in its free form, this Ab has a stimulatory activity. The inventors observed, for the three Ab, increased II-6 secretion when the PBMCs are respectively incubated with the anti-CD56/ZZ-Tat_{22-57C22-37)S}, (FIG. 16A), anti-CD335/ZZ-Tat_{22-57C22-37)S}, (FIG. 16B) and anti-CD336/ZZ-Tat_{22-57C22-37)S} complexes (FIG. 16C). This data therefore indicates that molecular complexes targeting NK cells and HSPGs can induce effective activation of immune cells.

### Example 14: Molecular Complexes Targeting Immune Checkpoints (ICP) Induce in Vitro the Secretion of IL-6 by Cells of the Immune System

### Materials and Methods

In order to prepare molecular complexes targeting ICPs, three antibodies specific to molecules considered to be ICPs were used. The first Ab (BioXCell reference BE0190), referred to as anti-CTLA-4 Ab, targets the CTLA-4 molecule. The second Ab (BioXCell reference BE0285), referred to as anti-PD-L1 Ab, targets the PD-L1 molecule. The third Ab (R&D reference MAB10542), referred to as anti-OX40 Ab, targets the OX40 molecule.

Each of these three Ab (30 nM for each molecule) was incubated in the absence or presence of ZZ-Tat_{22-57C22-37)S} or ZZ (5 nM for each molecule), for 24 hours at 4° C. in RPMI medium 5% human AB serum. The capacity of the ZZ region to bind the Fc region of these Ab made it possible to form different non-covalent molecular complexes, referred to as anti-CTLA-4/ZZ-Tat_{22-57C22-37)S}, anti-PD-L1/ZZ-Tat_{22-57C22-37)S}, anti-OX40/ZZ-Tat_{22-57C22-37)S}, anti-CTLA-4/ZZ, anti-PD-L1/ZZ, and anti-OX40/ZZ, respectively.

Human PBMCs were resuspended at 5×10⁶ cells/ml in RPMI medium 5% human AB serum. 100 μl of the cell suspension was distributed in a 96-well plate in the presence or absence of free Ab, ZZ or ZZ-Tat_{22-57C(22-37)S} and also the molecular complexes. After 24 h of incubation, the supernatants were collected for ELISA assay of the cytokine IL-6 carried out according to the manufacturer's instructions (R&D #DY406-05).

### Results

The inventors wondered if molecular complexes targeting ICPs could induce the activation of cells of the immune system. Since cell activation can lead to cytokine secretion, the inventors decided to evaluate in vitro the presence of IL-6. They did not find any in the supernatants resulting from incubation of PBMCs with the three different free Ab or with free ZZ, indicating that these compounds cannot induce activation when they are in the free form (FIGS. 17A, 17B, and 17C). In contrast, they found this cytokine in supernatants resulting from incubation with ZZ-Tat_{22-57C22-37)S}, demonstrating that the association of the double Ig-binding domain and the Tat_{22-57C22-37)S} HS-binding domain enables the cells of the system to be activated. However, the inventors observed that 11-6 secretion is further increased when the PBMCs are respectively incubated with the anti-CTLA-4/ZZ-Tat_{22-57C22-37)S}, (FIG. 17A), anti-PD-L1/ZZ-Tat_{22-57C22-37)S}, (FIG. 17B) and anti-OX40/ZZ-Tat_{22-57C22-37)S} complexes (FIG. 17C). In contrast, IL-6 is absent from the superatants resulting from incubation with anti-CTLA-4/ZZ, anti-PD-L1/ZZ, or anti-OX40/ZZ, indicating that the double ZZ domain does not contribute to activation mediated by these Ab. All this data therefore indicates that molecular complexes targeting ICPs and HSPGs can induce activation of immune cells.

1-16. (canceled)

17. An immunomodulatory composition, comprising at least one molecular complex,
wherein said complex consists of at least one ligand of a sulfated sugar of the glycosaminoglycan family (first ligand) and at least one ligand of a surface molecule of antigen-presenting cells or of NK or NKT cells (second ligand), and
wherein said ligands are bonded to one another and said complex is free of a specific antigen of the disease to be treated,
and at least one pharmaceutically acceptable carrier, a carrier substance and/or an adjuvant.

18. The composition according to claim 17, wherein the first ligand is a heparan sulfate-binding peptide selected from the group consisting of: a peptide derived from the HIV Tat protein, comprising at least the basic region Tat49-57 (SEQ ID NO: 3) such as the peptides Tat49-57 (SEQ ID NO: 3), Tat37-57 (SEQ ID NO: 8) and Tat22-57C_{(22-37)S} (SEQ ID NO:9); an R7 to R11 polyarginine peptide; and a peptide comprising the R domain of the diphtheria toxin (SEQ ID NO: 5) or at least the fragment DT453-467 (SEQ ID NO 7) of said domain, comprising the heparan sulfate binding region.

19. The composition according to claim 17, wherein the second ligand targets a surface molecule of antigen-presenting cells selected from the group consisting of: C-type lectin receptors, membrane-bound immunoglobulins, receptors for the constant region of immunoglobulins, and immune checkpoint molecules and ligands thereof.

20. The composition according to claim 17, wherein the second ligand is selected from the group consisting of: (i) antibodies directed against said surface molecules of antigen-presenting cells or of NK or NKT cells and fragments thereof containing at least the paratope; (ii) immunoglobulins, and fragments thereof comprising at least the Fc region; and (iii) proteins and protein fragments which bind to the Fc and/or Fab region of the antibodies.

21. The composition according to claim 20, wherein the immunoglobulins are IgG.

22. The composition according to claim 20, wherein the proteins and protein fragments which bind to the Fc and/or Fab region of the antibodies are selected from the group consisting of: protein A of S. aureus, the BB fragment thereof (SEQ ID NO: 1) and the ZZ derivative thereof (SEQ ID NO: 2).

23. The composition according to claim 17, wherein the at least one molecular complex is in the form of oligomers or a mixture of monomers and oligomers.

24. The composition according to claim 17, wherein the at least one molecular complex consists of a fusion protein between the first and the second ligand.

25. The composition according to claim 24, wherein the fusion protein comprises a first ligand selected from: Tat49-57 (SEQ ID NO: 3), Tat37-57 (SEQ ID NO: 8), Tat22-57C(22-37)S (SEQ ID NO 9), the R domain of the diphtheria toxin (SEQ ID NO: 5) or the fragment DT453-467 (SEQ ID NO 7) and a second ligand selected from: the BB fragment of the protein A (SEQ ID NO: 1) or the ZZ derivative thereof (SEQ ID NO: 2).

26. The composition according to claim 17, wherein the second ligand is an antibody or an antibody fragment and the first ligand forms a fusion protein with an immunoglobulin-binding element.

27. The composition according to claim 26, wherein said fusion protein comprises a first ligand selected from Tat49-57 (SEQ ID NO: 3), Tat37-57 (SEQ ID NO: 8), Tat22-57C(22-37)S (SEQ ID NO 9), the R domain of the diphtheria toxin (SEQ ID NO: 5) or the fragment DT453-467 (SEQ ID NO 7) and an immunoglobulin-binding element selected from the BB fragment of the protein A (SEQ ID NO: 1) or the ZZ derivative thereof (SEQ ID NO: 2).

28. The composition according to claim 26, wherein said fusion protein comprises a first ligand selected from Tat49-57 (SEQ ID NO: 3), Tat37-57 (SEQ ID NO: 8), Tat22-57C(22-37)S (SEQ ID NO 9), the R domain of the diphtheria toxin (SEQ ID NO: 5) or the fragment DT453-467 (SEQ ID NO 7) and an immunoglobulin-binding element which is the BB fragment of the protein A (SEQ ID NO: 1) and said fusion protein is complexed to the second ligand which consists of a whole immunoglobulin.

29. The composition according to claim 26, wherein said fusion protein comprises a first ligand selected from Tat49-57 (SEQ ID NO: 3), Tat37-57 (SEQ ID NO: 8), Tat22-57C(22-37)S (SEQ ID NO 9), the R domain of the diphtheria toxin (SEQ ID NO: 5) or the fragment DT453-467 (SEQ ID NO 7), and an immunoglobulin-binding element selected from the BB fragment of the protein A (SEQ ID NO: 1) or the ZZ derivative thereof (SEQ ID NO: 2) and wherein said fusion protein is complexed to the second ligand which is selected from an anti-RFcgamma I, II and/or III, anti-DEC-205, anti-DC-SIGN, anti-CD74, anti-CD275, anti-CD56, anti-CD335, anti-CD336, anti-CTLA-4, anti-PD-L1, anti-OX40 antibody, or a fragment of the preceding antibodies comprising at least the paratope.

30. The composition according to claim 17, which is an immunostimulatory composition.

31. The composition according to claim 17, which is for activating antigen-presenting cells, for activating NK or NKT cells, and/or for activating the secretion of the cytokines IL-6 and/or IL-12

32. The composition according to claim 31, wherein the antigen-presenting cells are dendritic cells or monocytes.

33. The composition according to claim 17, wherein the adjuvant is a CpG oligodeoxynucleotide, polyinosinic-polycytidylic acid or a mixture of CpG oligodeoxynucleotide(s) and polyinosinic:polycytidylic acid, and/or the carrier substance is a nanoparticle.

34. The composition according to claim 17, further comprising at least one immune checkpoint inhibitor.

35. The composition according to claim 34, wherein the immune checkpoint inhibitor is chosen from an anti-PD-1, an anti-PDL-1 and an anti-CTLA4.

36. A method of immunotherapy of cancer or infectious diseases, comprising the administration to a subject in need thereof of a therapeutically effective amount of the composition according to claim 17.
